(19) United States
(12) Patent Application Publication
(10) Pub. No.: US 2008/0260722 A1
(43) Pub. Date: Oct. 23, 2008

(54) Method for High Efficiency Survival/Proliferation of Human Embyonic Stem Cells and Human Embryo Survival in Culture

(75) Inventors: Peter J. Donovan, Irvine, CA (US); April D. Pyle, Los Angeles, CA (US); Leslie F. Lock, Irvine, CA (US)

Correspondence Address: EDWARDS ANGELL PALMER & DODGE LLP, P.O. BOX 55874, BOSTON, MA, 02205, US

(73) Assignee: The Johns Hopkins University Johns Hopkins Technology Transfer, Baltimore, MD (US)

(21) Appl. No.: 11/794,455

(22) PCT Filed: Dec. 30, 2005
(86) PCT No.: PCT/US05/47481

§ 371 Date: Apr. 28, 2008

Related U.S. Application Data
(60) Provisional application No. 60/640,692, filed on Dec. 30, 2004.
(60) Provisional application No. 60/675,520, filed on Apr. 28, 2005.

(51) Int. Cl. A61K 39/395; C12N 5/06; A61P 43/00; A61B 17/435

(52) U.S. Cl. 424/130.1; 435/404; 435/384; 435/375; 600/34

(57) ABSTRACT

The present invention provides a role for neurotrophins in hES cell survival and important new insights into the molecular mechanisms controlling the growth of these cells. Although previous studies identified growth factors that affect self-renewal of hES cells, the novelty of the present invention is the identification of factors that act through specific receptors present on hES cells and activate the receptors at physiological concentrations to promote survival and proliferation.

### CROSS REFERENCE TO RELATED APPLICATIONS

This application claims priority to U.S. Provisional Patent Application Ser. No. 60/640,692, filed Dec. 30, 2004, and U.S. Provisional Patent Application Ser. No. 60/675,520, filed Apr. 28, 2005, the entire disclosures of which both are incorporated herein by reference in their entirety.

### GOVERNMENT RIGHTS

This invention was made in part with government support under HD041553-03 and HL074596-03, both awarded by the National Institutes of Health. The government has certain rights to this invention.

### FIELD OF THE INVENTION

The invention relates to methods for culturing human embryonic stem (hES) cells and human embryos. More particularly, this invention relates to methods for increasing the efficiency of hES survival and proliferation, as well as growth of human embryos.

### BACKGROUND OF THE INVENTION

Human embryonic stem (hES) cells are pluripotent stem cells that have the dual abilities to self-renew as well as differentiate into all the cell types present in the body^{1 2 3}. As such they represent a potentially important renewable resource for the treatment of human disease. Differentiated cells derived from hES cells could be used to treat a wide variety of human disorders and diseases. Growth and expansion of pluripotent hES cells require a balance between survival, proliferation and self-renewal signals. Although some of the growth factors thought to be involved in self-renewal of murine and human ES cells have been identified, factors regulating human ES cell survival have yet to be identified. Basic Fibroblast Growth Factor (bFGF or FGF2) at high concentration maintains hES cells in an undifferentiated state and has a profound effect on hES cell self-renewal⁴. Clonal lines of hES cells have been established in the presence of bFGF, albeit at low efficiency (<1%)⁵. The BMP antagonist Noggin can synergize with bFGF to promote hES cell self-renewal and sustain hES cell proliferation⁴. In addition, the Wnt/β-catenin and Activin/TGFα pathways may also be important for maintaining pluripotency⁶⁻⁸. Despite the importance of these findings, the clonal growth of hES cells is poorly sustained even in the presence of bFGF and Noggin⁴. To maintain hES cells in bFGF and Noggin, the cells must be passaged as clumps either by scraping manually or treating with collagenase. Significantly, the survival of single hES cells is extremely low suggesting that factors that affect survival of hES cells are limiting in the culture conditions currently used, a fact that limits the ability to rapidly expand hES cell populations and to carry out many methods of genetic selection. Identification of factors that promote survival of hES cells could profoundly impact our understanding of the molecular mechanisms regulating hES cell growth and our ability to manipulate hES cells for therapeutic purposes.

Related to our ability to promote survival and proliferation of embryonic stem cells, there is a major problem with in vitro fertilization in that many human embryos do not survive or grow well in culture. Consequently, many embryos that are produced in the in vitro clinics do not develop and patients have to donate more eggs or sperm in order to develop new embryos. This is because very little is known about factors that are required for normal human embryo survival, growth or normal differentiation. Again, identification of factors that would promote survival and growth in embryos produced by in vitro fertilization would be of great benefit.

Throughout this application, various publications are referenced to by numbers. Full citations for these publications may be found at the end of the specification immediately following the Abstract. Other publications are parenthetically referenced within the text of the specification. The disclosures of these publications in their entireties are hereby incorporated by reference into this application in order to more fully describe the state of the art to those skilled therein as of the date of the invention described and claimed herein.

### SUMMARY OF THE INVENTION

Growth of hES cells as a pluripotent population requires a balance between survival, proliferation and self-renewal signals. Demonstrated herein is that TRK receptors, which mediate anti-apoptotic signals, are expressed by hES cells. The present invention provides that TRK ligands, brain derived neurotrophic factor (BDNF), neurotrophin 3 (NT-3) and neurotrophin 4 (NT-4) are survival factors for hES cells. Addition of neurotrophins to hES cell cultures effects a 36-fold improvement in their clonal survival. hES cell cultures maintained in neurotrophins remain diploid and retain full developmental potency. In the presence of neurotrophins, TRKs are phosphorylated in hES cells and TRK inhibition leads to hES cell apoptosis. The PI-3K pathway, but not the MAPK pathway, mediates survival activity of neurotrophins in hES cells. Neurotrophins improve hES cell survival and may facilitate methodologies for their manipulation as well as for development of high-throughput screens to identify factors responsible for hES cell differentiation.

More specifically, the present invention relates to an aqueous composition for culturing hES cells in vitro comprising a culture medium supplemented with added exogenous neurotrophins in an effective concentration to increase the survival of the hES cells cultured in the neurotrophin supplemented culture medium, relative to the survival of hES cells cultured in an unsupplemented culture medium. In preferred embodiments of the invention the neurotrophins include brain-derived neurotophic factor (BDNF), neurotrophin-3 (NT-3) and neurotrophin-4 (NT-4).

Another embodiment of the present invention is a method of culturing hES cells comprising in vitro culturing the stem cells in an aqueous composition comprising a culture medium supplemented with added exogenous neurotrophins in an effective concentration to increase the survival of the hES cells cultured in the neurotrophin supplemented culture medium, relative to the survival of hES cells cultured in an unsupplemented culture medium. In preferred embodiments of the invention the supplemented neurotrophins are selected from the group consisting of brain-derived neurotophic factor (BDNF), neurotrophin-3 (NT-3) and neurotrophin-4 (NT-4). This method provides for increased survival and proliferation of hES cells in culture.

Still another embodiment of the present invention includes an aqueous composition for culturing primate embryos in vitro comprising a culture medium capable of supporting embryo development supplemented with exogenous neurotrophins in an effective concentration to increase the survival of viable primate embryos cultured in the neurotrophin supplemented culture medium, relative to the survival of embyros cultured in an unsupplemented culture medium. Preferred embodiments of the invention include supplemented neurotrophins selected from the group consisting of brain-derived neurotophic factor (BDNF), neurotrophin-3 (NT-3) and neurotrophin-4 (NT-4).

Another embodiment of the present invention is a method of increasing the survival of a primate embryo following in vitro fertilization comprising incubating the embryo in a culture media containing neurotrophins. Again, preferred embodiments of the invention include neurotrophins selected from the group consisting of brain-derived neurotophic factor (BDNF), neurotrophin-3 (NT-3) and neurotrophin-4 (NT4).

A further embodiment of the invention includes a method of carrying out in vitro fertilization of a primate oocyte and achievement of pregnancy in a receptive female according to the following steps: inducing hyperovulation by hormonal therapy, retrieving and incubating the oocytes in vitro in a culture media, fertilizing the oocytes with freshly obtained, capacitated sperm to obtain primate embryos, incubating the primate embryos in vitro in a culture media supplemented with neurotrophins in an effective concentration to increase survival from the time of double pronuclei visualization to the implantation stage of the mature blastocyst, harvesting the blastocyst from the culture media and releasing it into a physiologically receptive uterus. Preferred embodiments include human primates.

One other embodiment of the present invention includes a method for arresting embryo development or contraception in primates in which a neurotrophin antagonist is administered intravenously, intramuscularly, or transdermally in a dose sufficient to decrease embryo survival. Again, preferred embodiments include human primates and neurotrophin antagonist which can be an antibody directed to a neurotrophin selected from the group consisting of brain-derived neurotophic factor (BDNF), neurotrophin-3 (NT-3) and neurotrophin-4 (NT-4).

Thus, the compositions and methods of the present invention provide for increased survival and proliferation of hES cells which will further allow their use in techniques including homologous recombination, gene trapping, growth factor screening, cDNA expression library screening and chemical screening. Moreover, because the methods utilize very few cells, such screening protocols can now be carried out rapidly (within 2-5 days) and at very high throughput. Currently, such screens are impossible to carry out using hES cells.

Also, the compositions and methods of the present invention provide for increased success involving in vitro fertilization, as well as contraception.

Other aspects of the invention are disclosed infra.

### BRIEF DESCRIPTION OF THE FIGURES

The objects and features of the invention can be better understood with reference to the following detailed description and accompanying figures.

FIG. 1. Human embryonic stem (hES) cells express members of the TRK receptor tyrosine kinase family of neurotrophin receptors; a. RT-PCR analysis of TRKA, TRK B, TRKC and p75^{NGFR} expression in H1 hES cells. Lane 1=molecular weight markers, Lane 2=TRKA, Lane 3=TRKB, Lane 4=TRKC, Lane 5=p75, Lane 6=no RT, Lane 7=beta-Actin; b. Immunofluorescence analysis of TRKA, TKB, TRKC, p75^{NGFR}, and OCT4 expression by H1 hES cells; c. Western analysis of TRKA, TRK B, TRKC and p75^{NGFR} expression in H1 and H9 hES cells; in each panel, lane 1=H1 or H9 hES cells, Lane 2=PC12 or LNCaP positive control; Lane 3=MEF negative control; Beta-tubulin was used as a loading control for all samples.

FIG. 2. Neurotrophins support both clonal survival of hES cells and their growth as diploid pluripotent stem cells; a. H1 and H9 hES cells were plated on MEFs at single-cell density in hES cell medium containing 50 ng/ml each BDNF, NT3 and NT4. hES colonies were counted after visualization by alkaline phosphatase staining; b. hES cells grown in neurotrophins retain the characteristics of pluripotent hES cells for at least 20 passages; Colony morphology remained normal in NTs (see colony at 10× and 20×); Clones established in NTs retained a diploid karyotype; Out of 106 karyograms analyzed, all were diploid; hES cells maintained in NTs express the POU-domain transcription factor Oct4 and the stem cell marker SSEA4; the middle panels show cells that were counterstained with DAPI. hES cells maintained in NTs formed well-differentiated teratomas when injected into NOD/Scid mice; Sections of teratomas stained with hematoxylin and eosin are shown in the bottom panels.

FIG. 3. Neurotrophins are expressed by mouse embryo fibroblasts (MEFs); a. RT-PCR analysis of NT3, NT4, BDNF, and NGF, expression by MEFs; b. Blocking antibodies to the neurotrophins (NT3, NT4, and BDNF) interfere with hES-cell survival activity of MEF-CM, whereas an NGF blocking antibody and an isotype-matched control IgY do not. Bars=standard deviation of n=3 experiments; Blocking NT activity is statistically significant at p<0.001 as compared to CM (*).

FIG. 4. Individual neurotrophins have dose-dependent effects on hES cell survival; a. Effect of NTs on hES cell survival at low-density on MEFs. hES cells were trypsinized and plated at low density (500 cells per well) on MEFs in 96-well plates in the presence of NT3, BDNF, NT4, NGF or hES medium lacking added growth factor; After 4-5 days, the colonies (comprised of approximately 7-11 cells) were fixed and stained for alkaline phosphatase; b. Effect of NTs on hES cell survival at low-density on Matrigel. hES cells survive poorly in hES medium alone either on MEFs (a.) or on Matrigel (b.) but show dose-dependent survival in the presence of the neurotrophins on MEFs (a) or on Matrigel (b); Bar=standard deviation of n=3 experiments; *All NTs showed statistically significant effects on hES cell survival at p<0.01 as compared to hES medium alone. NTs (BDNF, NT4 and NT3) also have statistically significant differences between concentrations of 0.01-100 ng/ml at p<0.05; c. Phase contrast images (at 4×) of morphology of hES cell colonies observed on MEFs or Matrigel™ in the low-density survival assay.

FIG. 5. hES cell TRK receptors undergo phosphorylation in response to neurotrophins; a. Immunoprecipitation of TRK receptors from hES cells. hES cell lysates were immunoprecipiated with a TRKB or TRKC antibody and probed with an anti-phosphotyrosine antibody, A 145kDa band was identified in hES cells. Beta-tubulin was used as a loading control to ensure equal starting amounts of protein before IP; b. Localization of phosphorylated TRK 490 in hES cells in the presence of NTs and upon withdrawal of NTs. hES cells grown in the presence of NTs for 24 hours were stained with P-TRK 490 antibody directed against a phosphorylated epitope present in the TRK receptors (red) and nuclei stained with DAPI (blue); Anti-P-TRK 490 antibody staining was fairly uniform in cells throughout the colonies (left panel). In hES cells in which the NTs were removed for 20 minutes, cells began to lose P-TRK 490.

FIG. 6. TRK signaling and survival can be blocked by pharmacological inhibition; TRK inhibitors were added to hES cells plated with MEF-CM in the low-density survival assay; hES cells were exposed to increasing concentrations of a GW441756, K252A, or vehicle (DMSO). Bars=standard deviation of n=3 experiments; *Trk inhibitors are statistically significant at p<0.008 as compared to CM.

FIG. 7. Loss of the TRK signaling pathway leads to hES cell death; a. TUNEL analysis of hES cell apoptosis in the presence or absence of NTs; hES cells were grown in the presence or absence of neurotrophins and apoptosis assayed by TUNEL analysis; More FITC-positive cells (green) were present in cells grown without NTs (right panel) as compared to cells grown in the presence of NTs (left panel); Counterstaining of nuclei in the same colonies with DAPI is shown in the lower panels; b. FACS analysis of apoptosis in hES cells grown in the presence or absence of NTs; After 24 hours of culture grown with or without NTs, hES cells were stained with fluorescein isothiocyanite-conjugated Annexin V and analyzed by flow cytometry, hES cells were identified by anti-SSEA-4 staining; hES cells grown in neurotrophins (left panel) had significantly fewer Annexin V-positive cells than their counterparts grown in normal hES cell culture medium (right panel).

FIG. 8. PI-3K activity is required for hES cell survival; a. Effect of inhibitors on the survival of hES cells; hES cells were plated as single cells at low-density in NTs but in the absence of bFGF; cells were plated in the presence or absence of increasing concentrations of inhibitors of PI-3K (LY294002), MAPK (PD98059) and STAT3 (Inhibitor peptide) as well as an inactive form of a MAPK kinase inhibitor (UO124) as a control; as further controls, hES cells were plated in hES cell medium; the effect of the PI-3K inhibitor LY294002 on hES cell survival was statistically different from the control (*) (p<0.006) at all concentrations tested; the STAT3, MAPK kinase, and UO124 control inhibitors had no effect on hES cell survival. bFGF was omitted from the media in all conditions tested; b. NTs have differential effects on AKT and MEK1/2 phosphorylation; hES cells were grown in the presence or absence of NTs (but without bFGF) and cell lysates probed with antibodies to phospho-AKT or phospho-MEK1/2; in the absence of NTs, a weak phospho-AKT band is observed; upon stimulation of hES cells with NTs a stronger band is observed; the intensity of the bands were compared to the total level of AKT and to a loading control (Beta-tubulin); in contrast no change in the level of phospho-MEK1/2 was observed by comparison with the controls.

FIG. 9. Expression of neurotrophins receptors on H9 hES cells and controls for antibody reactivity; a. RT-PCR of H9 hES cells with primers for TRKA, TRKB, TRKC and p75^{NGFR}. H9 cells express TRKB and TRKC but not TRKA; P75 is expressed at low levels; b. Lack of expression of TRKB and TRKC on MEFs or HeLa cells; MEFs and HeLa cells were fixed and stained with antibodies to TRKB and TRKC; no specific reactivity was observed; C. hES cells were stained with anti-TRKB antibody or with antibody that had been pre-incubated with either the specific immunogenic peptide or a nonspecific peptide; the immunogenic (specific) peptide blocked TRKB staining whereas the nonspecific peptide did not.

FIG. 10. Effect of neurotrophins on hES cell differentiation and TRK phosphorylation; a. hES cells grown in neurotrophins were stained with antibodies to neuronal markers: including GFAP, MAP2, Pax2 and Pax6; positive controls for antibodies included neuronal cell lines and primary neurons, glia and astrocytes; b. Staining of hES cell colonies with antibodies to phospho-TRK. In the presence of NTs, the anti-phospho-TRK antibody stains most of the cells in the colony (upper left panel); when NTs are removed from the culture medium for 20 minutes many of the cells lose phospho-TRK staining (upper middle panel); colonies grown in the presence of MEF-CM show phospho-TRK staining (upper right panel), suggesting that part of the survival activity of MEF-CM is due to NTs; colonies were counter-stained with DAPI to reveal cell nuclei (lower panels).

FIG. 11. Effect of neurotrophins on the population doubling time of hES cells; the population doubling time assay was performed essentially as described in Cowan C A et. al.³²; in short, approximately 20,000 hES cells were plated per well into 16 wells of a 24-well tissue culture plate containing either mitotically-inactivated MEFs or Matrigel; twenty four hours later, 4 wells of MEFs and 4 wells of hES cells (with or without NTs) plus MEFs or Matrigel were trypsinized to a single cell suspension and the cells from each well were counted with a Nucleocounter (New Brunswick Scientific, Edison, N.J.); the counts for MEFs were averaged and subtracted from the average of the counts corresponding to the wells with hES cells; this value was the baseline for the doubling time assay; this procedure was repeated at 48, 72, and 96 hours after seeding; analysis was performed using standard linear regression methods to calculate slope of the lines and R-squared (best fit) values.

### DETAILED DESCRIPTION OF THE INVENTION

The present invention provides a role for neurotrophins in hES cell survival and important new insights into the molecular mechanisms controlling the growth of these cells. Although previous studies identified growth factors that affect self-renewal of hES cells, the novelty of the present invention is the identification of factors that act through specific receptors present on hES cells and activate the receptors at physiological concentrations to promote survival and proliferation. Further, a physiological response, namely induction of apoptosis, results from blockade of the signaling pathway. The survival effect of NTs appears to be mediated through TRK activation of the PI-3K pathway. The ability of neurotrophins to support high-efficiency clonal survival of hES cells should facilitate many uses of hES cells that are currently difficult or impossible, such as genetic selection or high-throughput screening. The ability of neurotrophins to support hES cell survival has important implications for cell-based therapies.

### I. DEFINITIONS

When used in this specification, the following terms will be defined as provided below unless otherwise stated. All other terminology used herein will be defined with respect to its usage in the particular art to which it pertains unless otherwise noted.

As used herein, the use of the word “a” or “an” when used in conjunction with the term “comprising” in the claims and/or the specification may mean “one,” but it is also consistent with the meaning of “one or more,” “at least one,” and “one or more than one.”

“Basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves can not synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium that can be supplemented with bFGF, insulin, and ascorbic acid and which supports the growth of primate primordial stem cells in a substantially undifferentiated state can be employed.

“Conditioned medium” refers to a growth medium that is further supplemented with soluble factors derived from cells cultured in the medium. Techniques for isolating conditioned medium from a cell culture are well known in the art. As will be appreciated, conditioned medium is preferably essentially cell-free. In this context, “essentially cell-free” refers to a conditioned medium that contains fewer than about 10%, preferably fewer than about 5%, 1%, 0.1%, 0.01%, 0.001%, and 0.0001% than the number of cells per unit volume, as compared to the culture from which it was separated.

A “defined” medium refers to a biochemically defined formulation comprised solely of the biochemically-defined constituents. A defined medium may include solely constituents having known chemical compositions. A defined medium may also include constituents that are derived from known sources. For example, a defined medium may also include factors and other compositions secreted from known tissues or cells; however, the defined medium will not include the conditioned medium from a culture of such cells. Thus, a “defined medium” may, if indicated, include a particular compounds added to form the culture medium, up to and including a portion of a conditioned medium that has been fractionated to remove at least one component detectable in a sample of the conditioned medium that has not been fractionated. Here, to “substantially remove” of one or more detectable components of a conditioned medium refers to the removal of at least an amount of the detectable, known component(s) from the conditioned medium so as to result in a fractionated conditioned medium that differs from an unfractionated conditioned medium in its ability to support the long-term substantially undifferentiated culture of primate stem cells. Fractionation of a conditioned medium can be performed by any method (or combination of methods) suitable to remove the detectable component(s), for example, gel filtration chromatography, affinity chromatography, immune precipitation, etc. Similarly, or a “defined medium” may include serum components derived from an animal, including human serum components. In this context, “known” refers to the knowledge of one of ordinary skill in the art with reference to the chemical composition or constituent.

“Human Embryonic Stem cells” (hES cells) are pluripotent stem cells derived from a human embryo in the blastocyst stage, or human pluripotent cells produced by artificial means (such as by nuclear transfer) that have equivalent characteristics. Exemplary derivation procedures and features are provided in a later section.

hES cell cultures are described as “undifferentiated” when a substantial proportion (at least 20%, and possibly over 50% or 80%) of stem cells and their derivatives in the population display morphological characteristics of undifferentiated cells, distinguishing them from differentiated cells of embryo or adult origin. It is understood that colonies of undifferentiated cells within the population will often be surrounded by neighboring cells that are differentiated. It is also understood that the proportion of cells displaying the undifferentiated phenotype will fluctuate as the cells proliferate and are passaged from one culture to another. Cells are recognized as proliferating in an undifferentiated state when they go through at least 4 passages and/or 8 population doublings while retaining at least about 50%, or the same proportion of cells bearing characteristic markers or morphological characteristics of undifferentiated cells.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution.

“Feeder cells” are non-primordial stem cells on which stem cells, particularly primate primordial stem cells, may be plated and which provide a milieu conducive to the growth of the stem cells.

A cell culture is “essentially feeder-free” when it does not contain exogenously added conditioned medium taken from a culture of feeder cells nor exogenously added feeder cells in the culture, where “no exogenously added feeder cells” means that cells to develop a feeder cell layer have not been purposely introduced for that reason. Of course, if the cells to be cultured are derived from a seed culture that contained feeder cells, the incidental co-isolation and subsequent introduction into another culture of some small proportion of those feeder cells along with the desired cells (e.g., undifferentiated primate primordial stem cells) should not be deemed as an intentional introduction of feeder cells. Similarly, feeder cells or feeder-like cells that develop from stem cells seeded into the culture shall not be deemed to have been purposely introduced into the culture.

A “growth environment” is an environment in which stem cells (e.g., primate primordial stem cells) will proliferate in vitro. Features of the environment include the medium in which the cells are cultured, and a supporting structure (such as a substrate on a solid surface) if present.

“Growth factor” refers to a substance that is effective to promote the growth of stem cells and which, unless added to the culture medium as a supplement, is not otherwise a component of the basal medium. Put another way, a growth factor is a molecule that is not secreted by cells being cultured (including any feeder cells, if present) or, if secreted by cells in the culture medium, is not secreted in an amount sufficient to achieve the result obtained by adding the growth factor exogenously. Growth factors include, but are not limited to, basic fibroblast growth factor (bFGF), acidic fibroblast growth factor (aFGF), epidermal growth factor (GF), insulin-like growth factor-I (IGF-I), insulin-like growth factor-II (IGF-II), platelet-derived growth factor-AB (PDGF), and vascular endothelial cell growth factor (VEGF), activin-A, and bone morphogenic proteins (BMPs), insulin, cytokines, chemokines, morphogents, neutralizing antibodies, other proteins, and small molecules.

“Isotonic” refers to a solution having essentially the same tonicity (i.e., effective osmotic pressure equivalent) as another solution with which it is compared. In the context of cell culture, an “isotonic” medium is one in which cells can be cultured without an appreciable net flow of water across the cell membranes.

A solution having “low osmotic pressure” refers to a solution having an osmotic pressure of less than about 300 milli-osmols per kilogram (“mOsm/kg”).

“Neurotrophins” are a family of molecules that encourage survival of nervous tissue. Neurotrophic factors are secreted by cells in a neurons target field, and act by prohibiting the neuron from apoptosis. In this way, excess neurons are removed.

The neurotrophin family include nerve growth factors (NGF), Brain-derived neurotrophic factor (BDNF), neurotrophin 3 (NT-3), and neurotrophin 4 (NT-4). There are two classes of receptors, p75 and the Tyrosine kinases. p75 is a low affinity neurotrophin receptor, to which all neurotrophins bind. The Tyrosine kinases include TrkA, TrkB, and TrkC, and will only bind with specific neurotrophins, but with a much higher affinity.

Neurotrophins (NTs) are a family of polypeptide growth factors that control the apoptotic death or survival, growth, and differentiation of neurons. NTs also regulate several other cell populations such as lymphoid, epithelial, oligoglia, and mast cells. Disregulation of the NTs or their receptors plays a key role (etiological or upstream) in certain human pathologies. Hyperactivity may lead to inflammatory pain, or some forms of cancer by autocrine/paracrine growth. Loss of activity may lead to neurodegeneration, neuropathic pain, or some forms of cancer by absence of differentiation. Consequently the NTs and their receptors are important therapeutic targets, and pharmacological modulation may have applications ranging from treatment of chronic or acute neurodegeneration, some forms of cancer, and chronic pain (with agonists), and some forms of cancer or acute pain (with antagonists).

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “non-essential amino acid” refers to an amino acid species that need not be added to a culture medium for a given cell type, typically because the cell synthesizes, or is capable of synthesizing, the particular amino acid species. While differing from species to species, non-essential amino acids are known to include L-alanine, L-asparagine, L-aspartic acid, L-glutamic acid, glycine, L-proline, and L-serine.

A “primate-derived primordial stem cell” or “primate primordial stem cell” is a primordial stem cell obtained from a primate species, including humans and monkeys, and includes genetically modified primordial stem cells.

“Pluripotent” refers to cells that are capable of differentiating into one of a plurality of different cell types, although not necessarily all cell types. An exemplary class of pluripotent cells is embryonic stem cells, which are capable of differentiating into any cell type in the human body. Thus, it will be recognized that while pluripotent cells can differentiate into multipotent cells and other more differentiated cell types, the process of reverse differentiation (i.e., de-differentiation) is likely more complicated and requires “re-programming” the cell to become more primitive, meaning that, after re-programming, it has the capacity to differentiate into more or different cell types than was possible prior to re-programming.

A cell culture is “essentially serum-free” when it does not contain exogenously added serum, where no “exogenously added feeder cells” means that serum has not been purposely introduced into the medium. Of course, if the cells being cultured produce some or all of the components of serum, or if the cells to be cultured are derived from a seed culture grown in a medium that contained serum, the incidental co-isolation and subsequent introduction into another culture of some small amount of serum (e.g., less than about 1%) should not be deemed as an intentional introduction of serum.

“Substantially undifferentiated” means that population of stem cells (e.g., primate primordial stem cells) contains at least about 50%, preferably at least about 60%, 70%, or 80%, and even more preferably, at least about 90%, undifferentiated, stem cells. Fluorescence-activated cell sorting using labeled antibodies or reporter genes/proteins (e.g., enhanced green fluorescence protein [EGFP]) to one or more markers indicative of a desired undifferentiated state (e.g., a primordial state) can be used to determine how many cells of a given stem cell population are undifferentiated. For purposes of making this assessment, one or more of cell surface markers correlated with an undifferentiated state (e.g., Oct-4, SSEA-4, Tra-1-60, and Tra-1-81) can be detected. Telomerase reverse transcriptase (TERT) activity and alkaline phosphatase can also be assayed. In the context of primate primordial stem cells, positive and/or negative selection can be used to detect, for example, by immuno-staining or employing a reporter gene (e.g., EGFP), the expression (or lack thereof) of certain markers (e.g., Oct-4, SSEA-4, Tra-1-60, Tra-1-81, SSEA-1 (mouse ES cells), SSEA-3, nestin, telomerase, Myc, p300, and Tip60 histone acetyltransferases, and alkaline phosphatase activity) or the presence of certain post-translational modifications (e.g., acetylated histones), thereby facilitating assessment of the state of self-renewal or differentiation of the cells.

“Totipotent” refers to cells that are capable of differentiating into any cell type, including pluripotent, multipotent, and fully differentiated cells (i.e., cells no longer capable of differentiation into various cell types), such as, without limitation, embryonic stem cells, neural stem cells, bone marrow stem cells, hematopoietic stem cells, cardiomyocytes, neuron, astrocytes, muscle cells, and connective tissue cells.

### Human Embryonic Stem Survival in Culture

### Human Embryonic Stem Cells

Human embryonic stem (hES) cells can be isolated, for example, from human blastocysts obtained from human in vivo preimplantation embryos, in vitro fertilized embryos, or one-cell human embryos expanded to the blastocyst stage (Bongso, et al. (1989), Hum. Reprod., vol. 4: 706). Human embryos can be cultured to the blastocyst stage in G1.2 and G2.2 medium (Gardner, et al. (1998), Fertil. Steril., vol. 69:84). The zona pellucida is removed from blastocysts by brief exposure to pronase (Sigma). The inner cell masses can be isolated by immunosurgery or by mechanical separation, and are plated on mouse embryonic feeder layers, or in the defined culture system as described herein. After nine to fifteen days, inner cell mass-derived outgrowths are dissociated into clumps either by exposure to calcium and magnesium-free phosphate-buffered saline (PBS) with 1 mM EDTA, by exposure to dispase, collagenase, or trypsin, or by mechanical dissociation with a micropipette. The dissociated cells are then replated as before in fresh medium and observed for colony formation. Colonies demonstrating undifferentiated morphology are individually selected by micropipette, mechanically dissociated into clumps, and replated. Embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (about 200 U/mL), or by selection of individual colonies by mechanical dissociation, for example, using a micropipette.

Once isolated, the stem cells, e.g., hES cells, can be cultured in a culture medium according to the invention that supports the substantially undifferentiated growth of primate primordial stem cells using any suitable cell culturing technique. For example, a matrix laid down prior to lysis of primate feeder cells (preferably allogeneic feeder cells) or a synthetic or purified matrix can be prepared using standard methods. The primate primordial stem cells to be cultured are then added atop the matrix along with the culture medium. In other embodiments, once isolated, undifferentiated human embryonic stem cells can be directly added to an extracellular matrix that contains laminin or a growth-arrested human feeder cell layer (e.g., a human foreskin fibroblast cell layer) and maintained in a serum-free growth environment according to the culture methods of invention. Unlike existing human embryonic stem cell lines cultured using conventional techniques, human embryonic stem cells and their derivatives prepared and cultured in accordance with these methods can be used therapeutically since they will not have been exposed to animal feeder cells, feeder-cell conditioned media, or serum at some point of their life time, thereby avoiding the risks of contaminating human cells with non-human animal cells, transmitting pathogens from non-human animal cells to human cells, forming heterogeneous fusion cells, and exposing human cells to toxic xenogeneic factors.

Alternatively, the stem cells, e.g., primate primordial stem cells, can be grown on living feeder cells (preferably allogeneic feeder cells) using methods known in the cell culture arts. The growth of the stem cells is then monitored to determine the degree to which they have become differentiated, for example, using a marker for alkaline phosphatase or telomerase or detecting the expression of the transcription factor Oct-4, or by detecting a cell surface marker indicative of an undifferentiated state (e.g., in the context of human embryonic stem cells, a labeled antibody for any one or more of SSEA-4, Tra-1-60, and Tra-1-81). When the culture has grown to confluence, at least a portion of the undifferentiated cells is passaged to another culture vessel. The determination to passage the cells and the techniques for accomplishing such passaging can be performed in accordance with the culture methods of invention (e.g., through morphology assessment and dissection procedures).

Also alternatively, the cells are cultured in a culture vessel that contains a substrate selected from the group consisting of feeder cells, preferably allogeneic feeder cells, an extracellular matrix, a suitable surface and a mixture of factors that adequately activate the signal transduction pathways required for undifferentiated growth, and a solution-borne matrix sufficient to support growth of the stem cells in solution. Thus, in addition to the components of the solution phase of culture media of the invention, the growth environment includes a substrate selected from the group consisting of primate feeder cells, preferably allogeneic feeder cells, and an extracellular matrix, particularly laminin. Preferred: feeder cells for primate primordial stem cells include primate fibroblasts and stromal cells. In preferred embodiments, the feeder cells and stem cells are allogeneic. In the context of human embryonic stem cells, particularly preferred feeder cells include human fibroblasts, human stromal cells, and fibroblast-like cells derived from human embryonic stem cells. If living feeder cells are used, as opposed to a synthetic or purified extracellular matrix or a matrix prepared from lysed cells, the cells can be mitotically inactivated (e.g., by irradiation or chemically) to prevent their further growth during the culturing of primate primordial stem cells. Inactivation is preferably performed before seeding the cells into the culture vessel to be used. The primate primordial stem cells can then be grown on the plate in addition to the feeder cells. Alternatively, the feeder cells can be first grown to confluence and then inactivated to prevent their further growth. If desired, the feeder cells may be stored frozen in liquid nitrogen or at −140° C. prior to use. As mentioned, if desired such a feeder cell layer can be lysed using any suitable technique prior to the addition of the stem cells (e.g., primate stem cells) so as to leave only an extracellular matrix.

Not wishing to be bound to any theory, it is believed that the use of such feeder cells, or an extracellular matrix derived from feeder cells, provides one or more substances necessary to promote the growth of stem cells (e.g., primate primordial stem cells) and/or prevent or decrease the rate of differentiation of such cells. Such substances are believed to include membrane-bound and/or soluble cell products that are secreted into the surrounding medium by the feeder cells. Thus, those skilled in the art will recognize that additional cell lines can be used with the cell culture media of the present invention to equivalent effect, and that such additional cell lines can be identified using standard methods and materials, for example, by culturing over time (e.g., several passages) substantially undifferentiated primate primordial stem cells on such feeder cells in a culture medium according to the invention and determining whether the stem cells remain substantially undifferentiated over the course of the analysis. Also, because of the defined nature of the culture media provided herein, it is now possible to assay various compounds found in the extracellular matrix or secreted by feeder cells to determine their respective roles in the growth, maintenance, and differentiation of stem calls such as primate primordial stem cells.

When purified components from extracellular matrices are used in lieu of feeder cells, such components will include those provided by the extracellular matrix of a suitable feeder cell layer. Components of extracellular matrices that can be used include laminin, or products that contain laminin, such as MATRIGEL™, or other molecules that activate the laminin receptor and/or its downstream signaling pathway. Thus, for purposes of the invention, a molecule that activates the laminin receptor and/or its downstream signaling pathway in an analogous fashion to laminin (even with greater or reduced effectiveness, for example, having at least 25%, at least 50%, at least 75%, at least 100%, at least 150%, at least 300%, at lest 500%, or at least 5000% of activation activity per molecule as compared to a naturally occurring or recombinant form of laminin) shall be considered “laminin”, provided that it can be used in lieu of the laminin in a defined cell culture media for growing and maintaining primate primordial stem cells in a substantially undifferentiated state. MATRIGEL™ is a soluble preparation from Engelbreth-Holm-Swarm tumor cells that gels at room temperature to form a reconstituted basement membrane. Other extracellular matrix components include fibronectin, collagen, and gelatin. In addition, one or more substances produced by the feeder cells, or contained in an extracellular matrix produced by a primate feeder cell line, can be identified and used to make a substrate that obviates the need for feeder cells. Alternatively, these components can be prepared in soluble form so as to allow the growth and maintenance of undifferentiated of stem cells in suspension culture. Thus, this invention contemplates adding extracellular matrix to the fluid phase of a culture at the time of passaging the cells or as part of a regular feeding, as well as preparing the substrate prior to addition of the fluid components of the culture.

Any suitable culture vessel can be adapted to culture stem cells (e.g., primate primordial stem cells) in accordance with the invention. For example, vessels having a substrate suitable for matrix attachment include tissue culture plates (including multi-well plates), pre-coated (e.g., gelatin-pre-coated) plates, T-flasks, roller bottles, gas permeable containers, and bioreactors. To increase efficiency and cell density, vessels (e.g., stirred tanks) that employ suspended particles (e.g., plastic beads or other microcarriers) that can serve as a substrate for attachment of feeder cells or an extracellular matrix can be employed. In other embodiments, undifferentiated stem cells can be cultured in suspension by providing the matrix components in soluble form. As will be appreciated, fresh medium can be introduced into any of these vessels by batch exchange (replacement of spent medium with fresh medium), fed-batch processes (i.e., fresh medium is added without removal of spent medium), or ongoing exchange in which a proportion of the medium is replaced with fresh medium on a continuous or periodic basis.

### Applications

The defined cell culture media and methods for growing stem cells, particularly hES cells, in a substantially undifferentiated state in accordance with the present invention will be seen to be applicable to all technologies for which stem cell lines are useful. Of particular importance is the use of the instant cell culture media and methods of culturing, for example, primate primordial stem cells in screening to identify growth factors useful in culturing primate stem cells in an undifferentiated state, as well as compounds that induce such cells to differentiate toward a particular cell or tissue lineage. The instant invention also allows genetically modified stem cells to be developed, as well as the creation of new stem cell lines, especially new primate primordial stem cell lines. The establishment of new cell lines according to the invention includes normal stem cell lines, as well as abnormal stem cell lines, for example, stem cell lines that carry genetic mutations or diseases (e.g., stem cells infected with a pathogen such as a virus, for example, HIV). Cells produced using the media and methods of the invention can also be mounted on surfaces to form biosensors for drug screening. The invention also provides for the capacity to produce, for example, commercial grade undifferentiated primate primordial stem cells (hES) on a commercial scale. As a result, stem cells such as primate primordial stem cells produced in accordance with the present invention will have numerous therapeutic and diagnostic applications. In other applications, substantially undifferentiated hES can be used. Several representative examples of such applications are provided below.

### A. Screens for Growth Factors

An aspect of the present invention involves screens for identifying growth factors that promote or inhibit the differentiation, growth, or survival of stem cells such as primate primordial stem cells in serum-free, feeder-free culture, as well as factors that promote the differentiation of such cells. Such systems have the advantage of not being complicated by secondary effects caused by perturbation of the feeder cells by the test compounds. In some embodiments, primate primordial stem cells are used as a primary screen to identify substances that promote the growth of primate primordial stem cells in a substantially undifferentiated state. Such screens are performed by contacting the stem cells in culture with one test compound species (or, alternatively, pools of different test compounds). The effect of exposing the cells to the test compound can then be assessed using any suitable assay, including enzyme activity-based assays and reporter/antibody-based screens, e.g., to detect the presence of a marker correlated with an undifferentiated state. Such assays can be either qualitative or quantitative in terms of their read out. Suitable enzyme activity assays are known in the art (e.g., assays based on alkaline phosphatase or telomerase activity), as are antibody-based assays, any of which may readily be adapted for such applications. Of course, any other suitable assay may also be employed, depending on the result being sought.

With regard to antibody-based assays, polyclonal or monoclonal antibodies may be obtained that are specifically reactive with a cell surface marker that is correlated with totipotency or pluripotency. Such antibodies can be labeled. Alternatively, their presence may be detected by a labeled secondary antibody (e.g., a fluorescently labeled, rabbit-derived anti-mouse antibody that reacts with mouse-derived antibodies), as in a standard ELISA (Enzyme-Linked ImmunoSorbent Assay). If desired, labeled stem cells can also be sorted and counted using standard methods, e.g., fluorescence-activated cell sorting (“FACS”).

In one embodiment of such a primary screen, the presence of increased alkaline phosphatase activity (indicative of an undifferentiated state) indicates that the test compound is a growth factor. In other embodiments, increased percentages of cells with continued expression of one or more markers indicative of an undifferentiated state (e.g., Oct-4, SSEA-4, Tra-1-60, and Tra-1-81) following exposure to a test compound indicates that the test compound is a growth factor. Serial or parallel combinations of such screens (e.g., an alkaline phosphatase-based screen followed by, or alternatively coupled with, a screen based on expression of Oct-4, SSEA-4, Tra-1-60, and Tra-1-81) may also be employed. Substances that are found to produce statistically significant promotion of growth of the stem cells in an undifferentiated state can then be re-tested, if desired. They can also be tested, for example, against primordial stem cells from other primate species to determine if the growth factor exerts only species-specific effects. Substances found to be effective growth factors for primate stem cells can also be tested in combinations to determine the presence of any synergistic effects.

Such assays can also be used to optimize the culture conditions for a particular type of stem cell, such as primate primordial stem cells (hES).

In addition to screening for growth factors, stem cells cultured in accordance with the invention can also be used to identify other molecules useful in the continued culture of the cells in a substantially undifferentiated state, or alternatively, which stimulate a change in the developmental fate of a cell. Such changes in developmental fate include inducing differentiation of the stem cell toward a desired cell lineage. In other embodiments, the developmental change stimulated by the molecule may be de-differentiation, such that following exposure to the test compound, the cells become more primitive, in that subsequent to exposure, they have the capacity to differentiate into more cell types than was possible prior to exposure. As will be appreciated, such methods allow the evaluation of any compound for such an effect, including compounds already known to play important roles in biology, e.g., proteins, carbohydrates, lipids, and various other organic and inorganic molecules found in cells or which affect cells.

### B. Drug Screens

Feeder-free, serum-free cultures of stem cells such as hes cells can also be used in drug discovery processes, as well as for testing pharmaceutical compounds for potential unintended activities, as might cause adverse reactions if the compound was administered to a patient. Assessment of the activity of pharmaceutical test compounds generally involves combining the cells of the invention with the test compound, determining any resulting change, and then correlating the effect of the compound with the observed change. The screening may be done, for example, either because the compound is designed to have a pharmacological effect on certain cell types, or because a compound designed to have effects elsewhere may have unintended side effects. Two or more drugs (or other test compounds) can also be tested in combination (by combining with the cells either simultaneously or sequentially) to detect possible drug-drug interaction effects. In some applications, compounds are screened initially for potential toxicity. See generally “In vitro Methods in Pharmaceutical Research,” Academic Press, 1997. Cytotoxicity can be determined by the effect on cell viability, survival, morphology, on the expression or release of certain markers, receptors or enzymes, and/or on DNA synthesis or repair, measured by [.sup.3H]-thymidine or BrdU incorporation.

### C. Differentiated Cells

hES cells (or other stem cells) cultured according to this invention can be used to prepare populations of differentiated cells of various commercially and therapeutically important tissue types. In general, this is accomplished by expanding the stem cells to the desired number. Thereafter, they are caused to differentiate according to any of a variety of differentiation strategies. For example, highly enriched populations of cells of the neural lineage can be generated by changing the cells to a culture medium containing one or more neurotrophins (such as neurotrophin 3 or brain-derived neurotrophic factor), one or more mitogens (such as epidermal growth factor, bFGF, PDGF, IGF 1, and erythropoietin), or one or more vitamins (such as retinoic acid, ascorbic acid). Alternatively, multipotent neural stem cells can be generated through the embryoid body stage and maintained in a chemically defined medium containing bFGF. Cultured cells are optionally separated based on whether they express a nerve precursor cell marker such as nestin, Musashi, vimentin, A2B5, nurrl, or NCAM. Using such methods, neural progenitor/stem cells can be obtained having the capacity to generate both neuronal cells (including mature neurons) and glial cells (including astrocytes and oligodendrocytes). Alternatively, replicative neuronal precursors can be obtained that have the capacity to form differentiated cell populations.

Cells highly enriched for markers of the hepatocyte lineage can be differentiated from primate primordial stem cells by culturing the stem cells in the presence of a histone deacetylase inhibitor such as n-butyrate. The cultured cells are optionally cultured simultaneously or sequentially with a hepatocyte maturation factor such as EGF, insulin, or FGF.

hES cells can also be used to generate cells that have characteristic markers of cardiomyocytes and spontaneous periodic contractile activity. Differentiation in this way is facilitated by nucleotide analogs that affect DNA methylation (such as 5-aza-deoxy-cytidine), growth factors, and bone morphogenic proteins. The cells can be further enriched by density-based cell separation, and maintained in media containing creatine, carnitine, and taurine.

Additionally, stem cells such as primate primordial stem cells can be directed to differentiate into mesenchymal cells in a medium containing a bone morphogenic protein (BMP), a ligand for the human TGF-β receptor, or a ligand for the human vitamin D receptor. The medium may further comprise dexamethasone, ascorbic acid-2-phosphate, and sources of calcium and phosphate. In preferred embodiments, derivative cells have phenotypic features of cells of the osteoblast lineage.

As will be appreciated, differentiated cells derived from stem cells such as hES cells cultured in accordance with the methods of the invention can be also be used for tissue reconstitution or regeneration in a human patient in need thereof. The cells are administered in a manner that permits them to graft to the intended tissue site and reconstitute or regenerate the functionally deficient area. For instance, neural precursor cells can be transplanted directly into parenchymal or intrathecal sites of the central nervous system, according to the disease being treated. The efficacy of neural cell transplants can be assessed in a rat model for acutely injured spinal cord, as described by McDonald, et al. ((1999) Nat. Med., vol. 5:1410) and Kim, et al. ((2002) Nature, vol. 418:50). Successful transplants will show transplant-derived cells present in the lesion 2-5 weeks later, differentiated into astrocytes, oligodendrocytes, and/or neurons; and migrating along the spinal cord from the lesioned end, and an improvement in gait, coordination, and weight-bearing.

Similarly, the efficacy of cardiomyocytes can be assessed in a suitable animal model of cardiac injury or dysfunction, e.g., an animal model for cardiac cryoinjury where about 55% of the left ventricular wall tissue becomes scar tissue without treatment (Li, et al. (1996), Ann. Thorac. Surg., vol. 62:654; Sakai, et al. (1999), Ann. Thorac. Surg., vol. 8:2074; Sakai, et al. (1999), J. Thorac. Cardiovasc. Surg., vol. 118:715). Successful treatment will reduce the area of the scar, limit scar expansion, and improve heart function as determined by systolic, diastolic, and developed pressure (Kehat, et al. (2004)). Cardiac injury can also be modeled, for example, using an embolization coil in the distal portion of the left anterior descending artery (Watanabe, et al. (1998), Cell Transplant, vol. 7:239), or by ligation of the left anterior descending coronary artery (Min, et al. (2002), J. Appl. Physiol., vol. 92:288). Efficacy of treatment can be evaluated by histology and cardiac function. Cardiomyocyte preparations embodied in this invention can be used in therapy to regenerate cardiac muscle and treat insufficient cardiac function.

Liver function can also be restored by administering hepatocytes and hepatocyte precursors differentiated from, for example, primate pluripotent stem cells grown in accordance with this invention. These differentiated cells can be assessed in animal models for ability to repair liver damage. One such example is damage caused by intraperitoneal injection of D-galactosamine (Dabeva, et al. (1993), Am. J. Pathol., vol. 143:1606). Treatment efficacy can be determined by immunocytochemical staining for liver cell markers, microscopic determination of whether canalicular structures form in growing tissue, and the ability of the treatment to restore synthesis of liver-specific proteins. Liver cells can be used in therapy by direct administration, or as part of a bioassist device that provides temporary liver function while the subject's liver tissue regenerates itself, for example, following fulminate hepatic failure.

### D. Genetically Modified Primate Stem Cells

The present invention also provides methods for producing, for example, hES cell lines having one or more genetic modifications. As is apparent to one of ordinary skill in the art, altered expression of gene products can be achieved by modifying the coding sequence of a gene product or by altering flanking regions of the coding sequence. Thus, as used herein, the terms “genetic modification” and the like include alterations to the sequence encoding a gene product, as well as alterations to flanking regions, in particular to the 5′ upstream region of the coding sequence (including the promoter). Similarly, the term “gene” encompasses all or part of the coding sequence and the regulatory sequences that may be present flanking the coding sequence, as well as other sequences flanking the coding sequence. Genetic modifications may be permanent or transient. Preferred permanent modifications are those that do not adversely affect chromosome stability or cell replication. Such modifications are preferably introduced by recombination or otherwise by insertion into a chromosome (as may be mediated, for example, by an engineered retroviral vector). Transient modifications are generally obtained by introducing an extrachromosomal genetic element into a cell by any suitable technique.

Regardless of the permanence of a particular genetic modification, in embodiments wherein one or more genes are introduced, their expression may be inducible or constitutive. The design, content, stability, etc. of a particular genetic construct made for use in practicing the invention is left to the discretion of the artisan, as these will vary depending on the intended result.

After introducing a desired genetic modification, a particularly effective way of enriching genetically modified cells is positive selection using resistance to a drug such as neomycin. To accomplish this, the cells can be genetically altered by contacting them simultaneously with a vector system harboring the gene(s) of interest, and a vector system that provides the drug resistance gene. Alternatively, the drug resistance gene can be built into the same vector as the gene(s) of interest. After transfection has taken place, the cultures are treated with the corresponding drug, and untransfected cells are eliminated.

According to this aspect, genetically modified stem cells such as primate primordial stem cells are grown using a cell culture medium of the invention. One or more genes or nucleic acid molecules are introduced into, or one or more genes are modified in, these cells to produce a clone population having the desired genetic modifications. Depending upon the genetic modification(s) made, the cells may continue to be propagated in a substantially undifferentiated state in accordance with the invention. Alternatively, they may be allowed (or induced) to differentiate. Primate-derived primordial stem cells having such genetic modifications have important applications, especially with respect to applications where euploid primate cells having genetic modifications are useful or required. Examples of such applications include, but are not limited to, the development of cell-based models for primate, especially human, diseases, as well as the development of specialized tissues for transplantation. Genetically modified stem cells cultured in accordance with the invention, including primate primordial stem cells, especially hES cells, also have many other therapeutic applications, including in gene therapy (e.g., to compensate for a single gene defect), and as tissue for grafting or implantation, and to treat other diseases and disorders. Examples of diseases caused by single gene defects include myotonic dystrophy, cystic fibrosis, sickle cell anemia, Tay Sachs disease, and hemophilia.

For therapeutic application, cells prepared according to this invention (be they totipotent or pluripotent cells or differentiated cells derived there from) are typically supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells may be packaged in a device or container suitable for distribution or clinical use, optionally accompanied by information relating to use of the cells in tissue regeneration or for restoring a therapeutically important metabolic function.

### General Cell Culture Methods:

The present invention relies on routine techniques in the field of cell culture, and those with skill in the art can easily determine suitable conditions. In general, the cell culture environment includes consideration of such factors as the substrate for cell growth, cell density and cell contract, the gas phase, the medium, the temperature, and the presence of growth factors.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts useful include Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Invitrogen, Nalgene-Nunc International, Sigma Chemical Co., Chemicon International, and ICN Biomedicals.

Exemplary cell culture conditions for stem cells are described in, e.g., U.S. Pat. Nos. 6,562,619 and 6,875,607; and USPN's 20050158852; 20040180347; 20040224403; 20050064589; 20050233446; 20050244962; and 20030073234, each of which is incorporated by reference in their entirety.

The cells of the invention can be grown under conditions that provide for cell to cell contact. In a preferred embodiment, the cells are grown in suspension as three dimensional aggregates. Suspension cultures can be achieved by using, e.g., a flask with a magnetic stirrer or a large surface area paddle, or on a plate that has been coated to prevent the cells from adhering to the bottom of the dish. For example, the cells may be grown in Costar dishes that have been coated with a hydrogel to prevent them from adhering to the bottom of the dish.

For cells that grow in a monolayer attached to a substrate, plastic dishes, flasks, roller bottles, or microcarriers are typically used. Other artificial substrates can be used such as glass and metals. The substrate is often treated by etching, or by coating with substances such as collagen, chondronectin, fibronectin, laminin or poly-D-lysine. The type of culture vessel depends on the culture conditions, e.g., multi-well plates, petri dishes, tissue culture tubes, flasks, roller bottles, microcarriers, and the like. Cells are grown at optimal densities that are determined empirically based on the cell type.

Important constituents of the gas phase are oxygen and carbon dioxide. Typically, atmospheric oxygen tensions are used for dendritic cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media, and is typically present at a concentration of 1-10% in the incubator. The preferred CO₂ concentration for dendritic cell cultures is 5%.

Cultured cells are normally grown in an incubator that provides a suitable temperature, e.g., the body temperature of the animal from which is the cells were obtained, accounting for regional variations in temperature. Generally, 37° C. is the preferred temperature for dendritic cell culture. Most incubators are humidified to approximately atmospheric conditions.

Defined cell media are available as packaged, premixed powders or presterilized solutions. Examples of commonly used media include Iscove's media, RPMI 1640, DMEM, and McCoy's Medium (see, e.g., GibcoBRL/Life Technologies Catalogue and Reference Guide; Sigma Catalogue). Defined cell culture media are often supplemented with 5-20% serum, e.g., human, horse, calf, or fetal bovine serum. The culture medium is usually buffered to maintain the cells at a pH preferably from about 7.2 to about 7.4. Other supplements to the media include, e.g., antibiotics, amino acids, sugars, and growth factors (see, e.g., Lutz et al., supra).

### Embryo Survival In Vitro Fertilization and Contraception

The present invention also relates to a method for increasing the rate of implantation of primate embryos in a receptive uterus, and to certain media in which an embryo may be cultured in carrying out this method. A method is also disclosed for reducing the likelihood of embryo implantation, which may have contraceptive applications.

In particular, the present invention relates to an aqueous composition for culturing primate embryos in vitro comprising a culture medium capable of supporting embryo development supplemented with exogenous neurotrophins in an effective concentration to increase the survival of viable primate embryos cultured in the neurotrophin supplemented culture medium, relative to the survival of embyros cultured in an unsupplemented culture medium.

Further, the present method of increasing survival of primate embryos in vitro comprises culturing a fertilized primate morula, blastomere, or blastocyst in the presence of one or more neurotrophins in a physiologically sufficient concentration from the time of its suspension in culture media to the implantation stage of the blastocyst. A physiologically sufficient concentration (means a relevant in vivo concentration) means that amount necessary to achieve attachment of greater than 90 percent) of embryos on an empirical basis.

A further embodiment of the present invention provides for the foregoing embryo culture procedure together with the further steps of removing the blastocyst from culture prior to in vitro attachment and cell differentiation, and releasing it into a physiologically receptive uterus. Ordinarily the physiologically receptive uterus is that of the oocyte donor, which has been made receptive through the hormonal manipulations leading to ovulation. However, a hormonally primed surrogate may also be a recipient.

The present culture method is adaptable to in vitro fertilization of a primate oocyte and achievement of pregnancy in a receptive female according to conventional practice involving the steps of inducing hyperovulation by hormonal therapy, retrieving oocytes under laparoscopy or other standard or medically acceptable method, incubating the oocytes in vitro in a culture medium for a suitable period until the oocytes are cytologically mature for fertilization, fertlizing with freshly obtained or frozen, stored, newly capacitated sperm, incubating in enriched culture medium in the presence of one or more neurotrophin in a concentration sufficient to provide prolonged survival of viable primate embryos, incubation of the fertilized oocyte continuing from the time of double pronuclei visualization to the implantation stage of the mature blastocyst, harvesting the mature blastocyst from culture and releasing it into a physiologically receptive uterus.

The present invention also provides a culture medium containing one or more neurotrophin, either in final aqueous form containing protein supplements, or as a defined base medium in dry form, which can be reconstituted. The culture medium is an aqueous composition comprising a conventional culture medium supportive of embryo development supplemented with one or more neurotrophin. In a preferred embodiment, the aqueous composition for culturing primate embryos in vitro comprises a culture medium containing inorganic salts in a physiologically compatible range of concentration, essential L-amino acids in nutritive concentrations, essential vitamins in concentrations supportive of embryonic growth, purine and pyrimidine sources in physiologic concentration, energy generating system cofactors, buffering agents, a metabolizable carbon source, a physiologically compatible protein carrier solution, and one or more neurotrophin in a concentration sufficient to provide prolonged survival and enhanced attachment and implantation rates of viable primate embryos.

In dry form, the defined base culture medium comprises a powdered mixture containing inorganic salts, essential L-amino acids, essential vitamins, sources of purines and pyrimidines, energy generating systems cofactors, buffering agents, a metabolizable carbon source, all of which are present in such proportions that when reconstituted in aqueous solution are in physiologically compatible concentrations, together with lyophilized neurotrophin(s) present in a proportion that when the powdered mixture is fully reconstituted, it is present in a concentration sufficient to provide prolonged survival and enhanced attachment and implantation rates of viable primate embryos.

The present method also provides a method for preventing uterine implantation of an embryo in a primates in which a neurotrophin antagonist (including receptor and signal transduction pathway antagonists) is administered intravenously, intramuscularly, or transdermally in a dose sufficient to arrest embryo development. Neurotrophin antagonists include polyclonal and monoclonal antibodies and immunoreactive fragments thereof which are directed to any one of the factors in the neurotrophin family. Other neurotrophin antagonists include small peptides and small molecule mimetics.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXEMPLIFICATION

The following Examples are provided to illustrate certain aspects of the present invention and to aid those of skill in the art in practicing the invention. These Examples are in no way to be considered to limit the scope of the invention in any manner.

General methods in molecular genetics and genetic engineering are described in the current editions of “Molecular Cloning: A Laboratory Manual” (Sambrook, et al., Cold Spring Harbor); Gene Transfer Vectors for Mammalian Cells (Miller & Calos eds.); and “Current Protocols in Molecular Biology” (Ausubel, et al. eds., Wiley & Sons). Cell biology, protein chemistry, and antibody techniques can be found in “Current Protocols in Protein Science” (Colligan, et al. eds., Wiley & Sons); “Current Protocols in Cell Biology” (Bonifacino, et al., Wiley & Sons) and “Current Protocols in Immunology” (Colligan et al. eds., Wiley & Sons.). Reagents, cloning vectors, and kits for genetic manipulation referred to in this disclosure are available from commercial vendors such as BioRad, Stratagene, Invitrogen, ClonTech, and Sigma-Aldrich Co.

### Materials and Methods

### Cell Culture, Survival Assays and Inhibitor Assays.

Human ES cell lines H1 and H9 were cultured as described by Thornson and colleagues¹⁵ in high glucose DMEM supplemented with L-glutanine, non-essential amino acids, serum replacement (Invitrogen) and 4 ng/ml basic Fibroblast growth factor (Invitrogen). Mouse embryo fibroblasts (MEFs) (Specialty Media). were plated at 5-7×10⁶ cells/ml. To prepare single cell suspensions of hES cells, the cells were washed twice with PBS, then incubated in 0.05% trypsin/EDTA (InVitrogen) for 5 minutes at 37° C. The cells were triturated with a 5-ml pipette until a single cell suspension was obtained. Trypsin inhibitor (InVitrogen) was added and the cell suspension filtered through a 0.4 mM filter (Fisher Scientific) to remove clumps and MEFs. For clonal survival assays, single hES cells were diluted to clonal density and plated into a well of a 96-well plate containing MEFs. For low-density survival assays, trypsinized cells were counted using a hemocytometer and 500 cells were plated in each well of 96-well plate containing MEFs or coated with Matrigel (BD Biosciences). To visualize hES colonies, cultures were fixed in 4% para-formaldehyde in PBS for 30 minutes, washed once in PBS, once in distilled H₂O, then stained for alkaline phosphatase activity as described previously²⁵. Individual hES cells or hES cell colonies were counted manually on an inverted microscope. NGF, BDNF, NT3 and NT4 were purchased from Peprotech. The TRK inhibitors GW441756 (Sigma) and K252A (Calbiochem), PI-3K inhibitor, MAPK inhibitors, STAT3 inhibitor and control peptides (all from Calbiochem) were prepared according to the manufacturer's instructions. For the antibody blocking experiments, antibodies to NGF, NT3, NT4 and BDNF as well as a control antibody were obtained from Promega. Antibodies were added to culture medium at a concentration of 20 ug/ml. Following culture, the cultures were fixed and stained for alkaline phosphatase activity as described above. Each experiment consisted of at least four replicates per treatment and each experiment was repeated at least three times. Statistical analysis was carried out using Students T-test analysis.

Immunocytochemistry. Immunocytochemical analysis of surface antigens was carried out as described previously 25. For staining of intracellular antigens, the cells were fixed in 4% paraformaldehyde in PBS, treated for 3 to 5 minutes with 0.05% Triton X-100 in PBS, then washed three times in PBS. Antibodies to SSEA-3, SSEA-4, Tra-1-60, Tra-1-81, Oct-4, TRKA, TRKC, and p75^{NGFR} were obtained from R and D Systems. Antibodies to Pax2 and Pax6 were obtained from Covance, MAP2 and GFAP were from Chemicon, and TRKB from Promega. Antibodies were diluted in blocking buffer containing 4% BSA and 10% heat-inactivated serum of the same species as the secondary antibody. Fluorescein- or rhodamine-conjugated secondary antibodies (Pierce) were diluted in the same blocking buffer. Positive controls for the reactivity of the neuronal antibodies (TRKA, TRKB, TRKC, p75^{NGFR}, Pax2, Pax6, MAP2 and GFAP) were primary rat neurons, astrocytes and glia as well as rat pheochromocytoma cells (PC12; generous gift from Dr. David Ginty). TUNEL staining was carried out using the ApopTag Plus FITC In Situ kit (Chemicon) according to the manufacturer's instructions. Peptide inhibition of antibody binding was carried out as described previously²⁸. Immunoctyochemical staining of cells was observed on a Nikon E1000 microscope equipped with fluorescence optics.

Immunoblotting and immunoprecipitation. Immunoblotting of hES cell antigens was carried out using published methods appropriate for each antibody. Antibodies to TRKA, TRKB, TRKC, p75^{NGFR} and the anti-phosphotyrosine monoclonal antibody 4G10 were obtained from Upstate. Lysates of MEFs were used as a negative control. Cells known to express the specific neurotrophin receptors were used as positive controls. PC12 express TRKA and p75^{NGFR} and human metastatic prostate carcinoma cells (LNCaP; ATCC) express TRKB and TRKC^{26,27}. PC12 cells were grown as described²⁸. LNCaP cells were grown according to ATCC instructions (see http://www.atcc.org/). Cell lysates were prepared as described previously²⁹. Immunoprecipitation of TRKB and TRKC in hES cell lysates was carried out as described by Kaplan and colleagues 30. Briefly, hES cells were treated with or without neurotrophins for 5 minutes, washed briefly in ice-cold PBS, then lysed in TBS plus 1% NP40, 10 ug/ml aprotinin, 1 mM PMSF, 1 mg/ml leupeptin and 500 uM Na Orthovanadate. Cells were rocked on ice and frozen and thawed three times. Lysates were centrifuged to remove debris, incubated with primary antibody overnight at 4° C., and then incubated with Protein-A-sepharose (Pharmacia) for 1-2 hours at 4° C. The beads were washed 3 times in lysis buffer, once in distilled H₂O, then incubated with SDS-PAGE sample buffer for 5 minutes at 90-100° C. The sample buffer was carefully removed and run on a 7.5% SDS-PAGE gel (BioRad) as described previously²⁹. Western blotting was carried out essentially as described previously²⁹. The resolved proteins were transferred overnight at 4° C. onto Immobilon PVDF membranes (Millipore) and then the PVDF membrane blocked in 1% BSA, 1% non-fat milk and 0.05% Tween 20 in PBS for 1-2 hours. Anti-P-Tyr antibody (4G10) was used according to the manufacturer's instructions. Primary antibodies were detected with species-specific horseradish peroxidase-conjugated secondary antibodies (Upstate) and a chemiluminescent kit (Cell Signaling).

Flow cytometry. Analysis of apoptosis in hES cell cultures was carried out by flow cytometry using Annexin-V-FITC (BD Biosciences) according to the manufacturer's instructions. hES cells were identified by staining with anti-SSEA-4 antibody (R and D Systems). Samples were analyzed on a FACSCalibur flow cytometer (Becton Dickinson).

Kartoytping and teratoma assays. Karyotype analysis was carried out routinely every 2 to 3 months as described previously³¹. For each analysis 10 to 20 karyograms were examined. Teratoma assays were carried out as described by Thomson and colleagues¹⁵. Briefly, hES cells from one nearly-confluent 6-well plate were harvested then injected subcutaneously into the hind limb or rear flank of a SCID/Beige mouse (The Jackson Labs). Animals were monitored regularly for signs of discomfort and distress. All animal work was carried out under protocols approved by the Johns Hopkins University IACUC. When tumors were visible, animals were sacrificed, the tumor material excised, then fixed and processed for histochemistry as described previously³¹. Differentiation of hES cells into embryoid bodies was carried out essentially as described by Thomson and colleagues

RT-PCR analysis. mRNA was prepared from hES cells or MEFs as previously described²⁹. Primers for the human TRKA, TRKB, TRKC and p75^{NGFR} genes as well as for the mouse NGF, NT3, NT4 and BDNF are described below. RT-PCR analysis was carried out using the Superscript One-Step RT-PCR with Platinum Taq Kit (InVitrogen) according to the manufacturer's instructions for 30-32 cycles. Amplification of β-actin or omission of RT served as positive and negative controls. The primers used for the individual genes were:

Human TRKA:
Forward primer:
TTC CAT TTC ACT CCT CGG CTC AGT
Reverse primer:
ACG TCA CGT TCT TCC TGT TGA GGT
Human TRKB
Forward primer:
TCA ATG CCA GGC AGG TCT CCT AAA
Reverse primer:
TTG GTG CAG AAT TCC CAG CAA AGG
Human TRKC
Forward primer:
TGC AGT CCA TCA ACA CTC ACC AGA
Reverse primer:
TGT AGT GGG TGG GCT TGT TGA AGA
Human p75^{NGFR}
Forward primer:
TTC AAG GGC TTA CAC GTG GAG GAA
Reverse primer:
TGT GTG TAA GTT TCA GGA GGG CCA
Mouse NT3
Forward primer:
CTT ATC TCC GTG GCA TCC AAG G
Reverse primer:
TCT GAA GTC AGT GCT CGG ACG T
Mouse NT4
Forward primer:
TTC TGG CTC CTG AGT GGA C
Reverse primer:
AGT CAA CGC CCG CAC ATA G
Mouse BDNF
Forward primer:
ATG GGA CTC TGG AGA GCG TGA A
Reverse primer:
CGC CAG CCA ATT CTC TTT TTG C
Mouse NGF
Forward primer:
GGT GCA TGG CGT AAT GTC CAT GTT
Reverse primer:
ATT GTA CCA TGG GCC TGG AAG TCT

The expected amplified products were: TRKA (463 bp), TRKB (493 bp), TRKC (372 bp), p75^{NGFR} (721 bp), NT3 (486 bp), NT4 (252 bp), BDNF (501 bp) and NGF (345 bp). For each of the genes the RT-PCR products were sequenced and verified by NCBI blast analysis.

### EXAMPLES

### Example 1

### Expression of Tyrosine Kinase by hES Cells

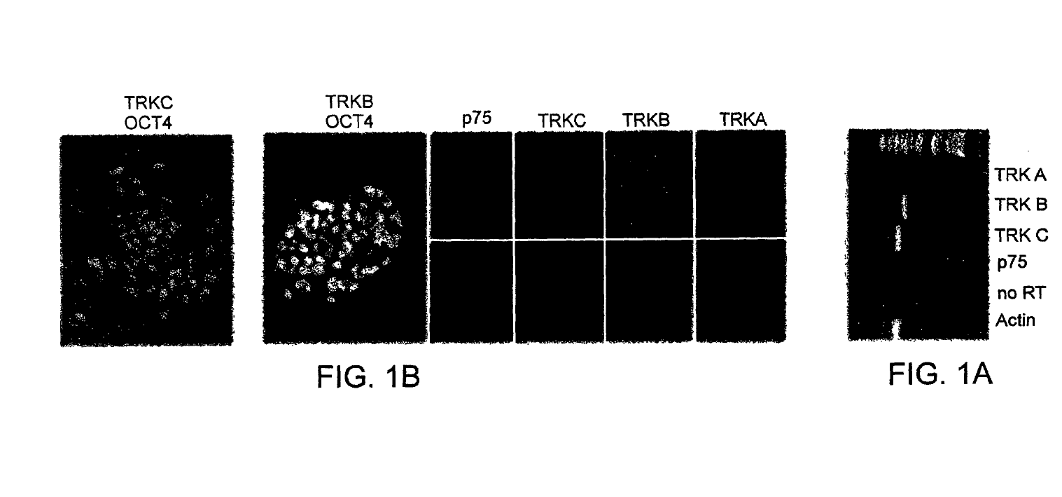

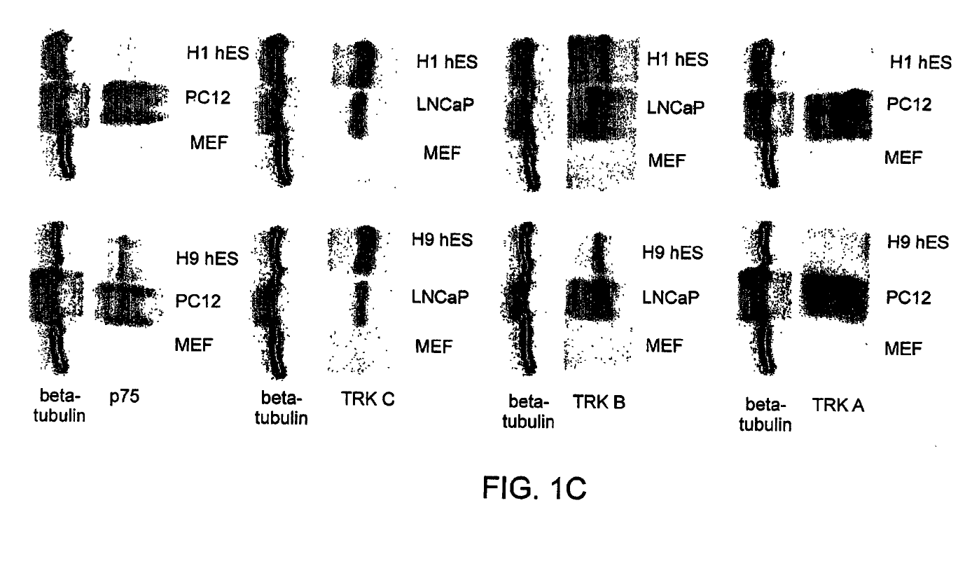

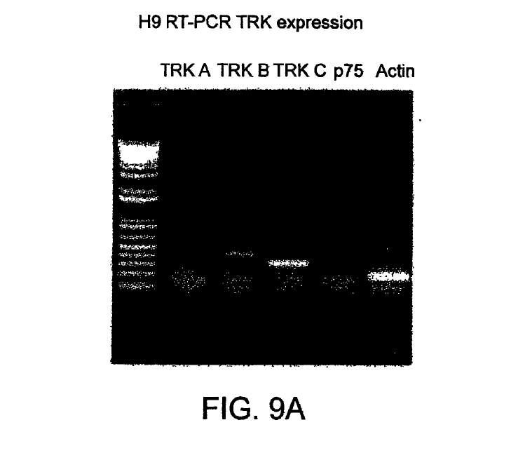

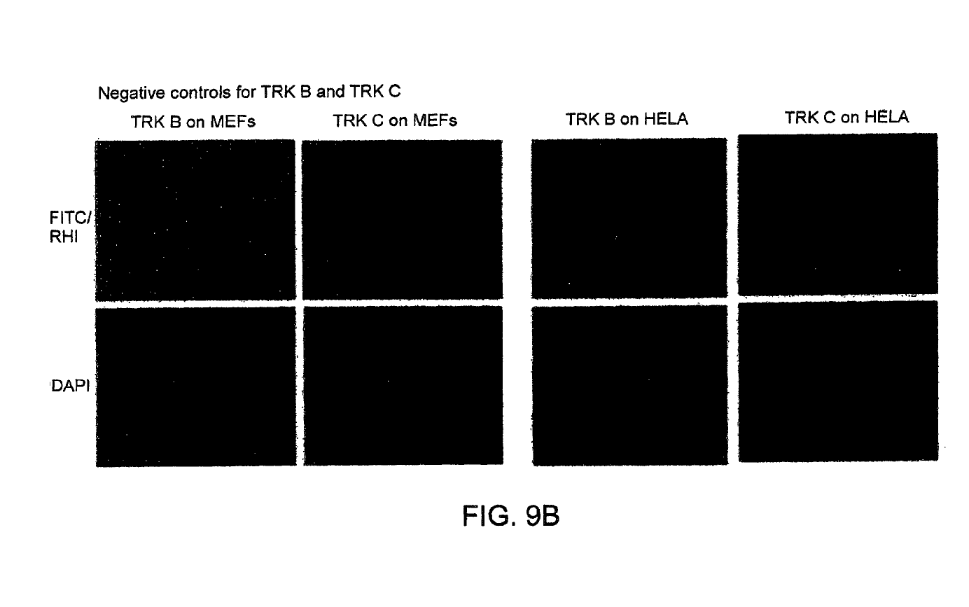

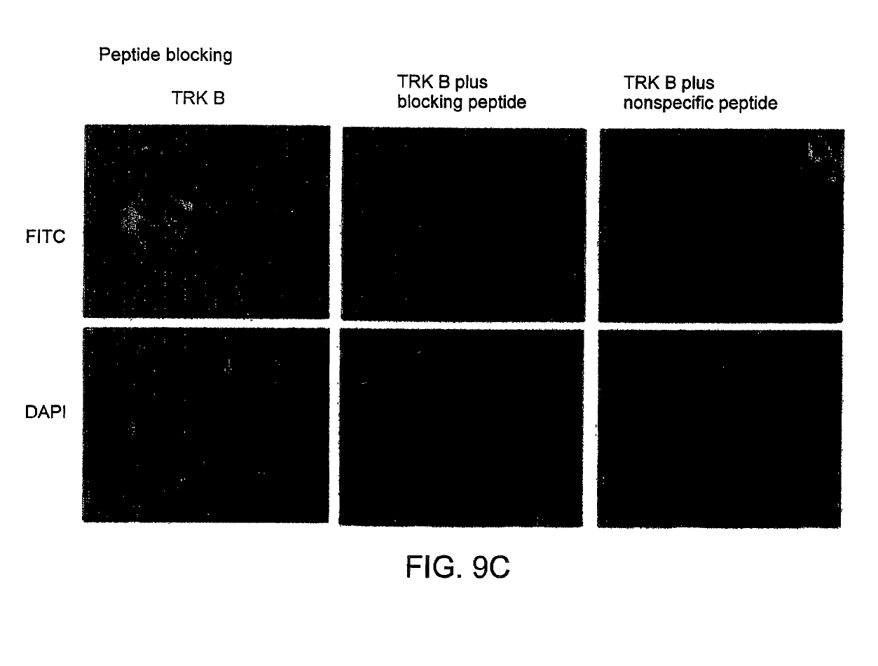

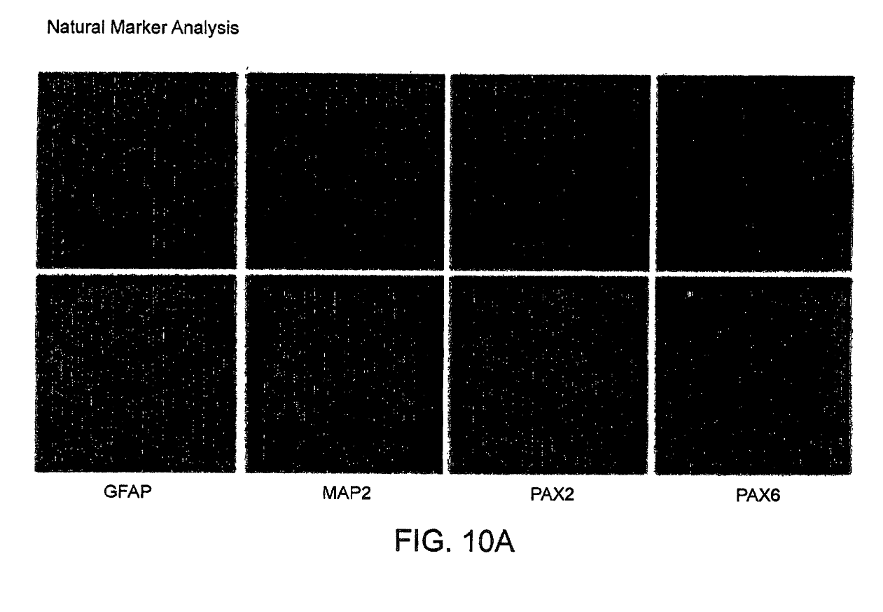

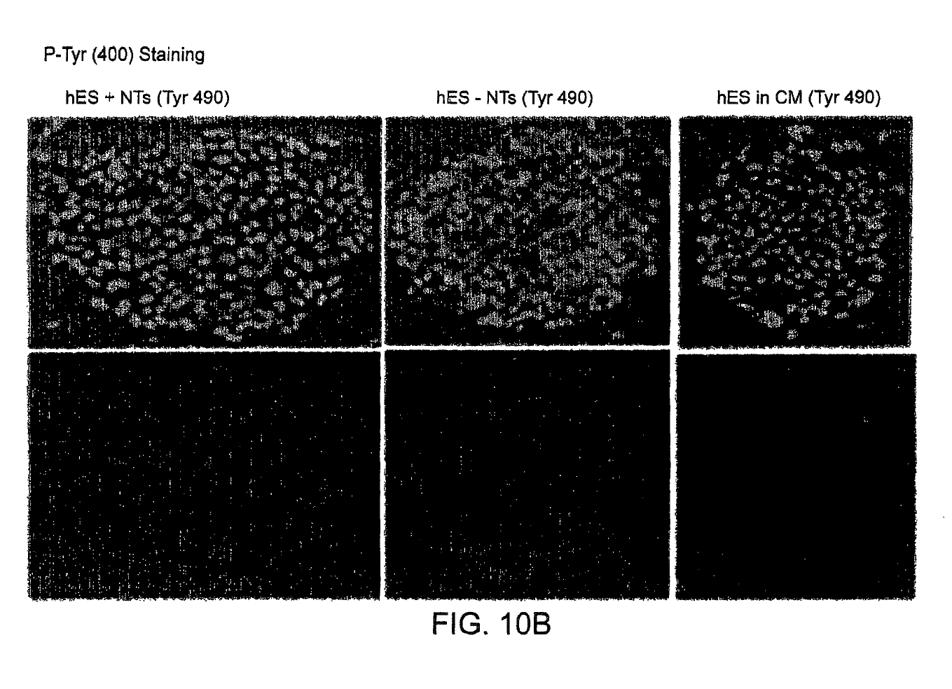

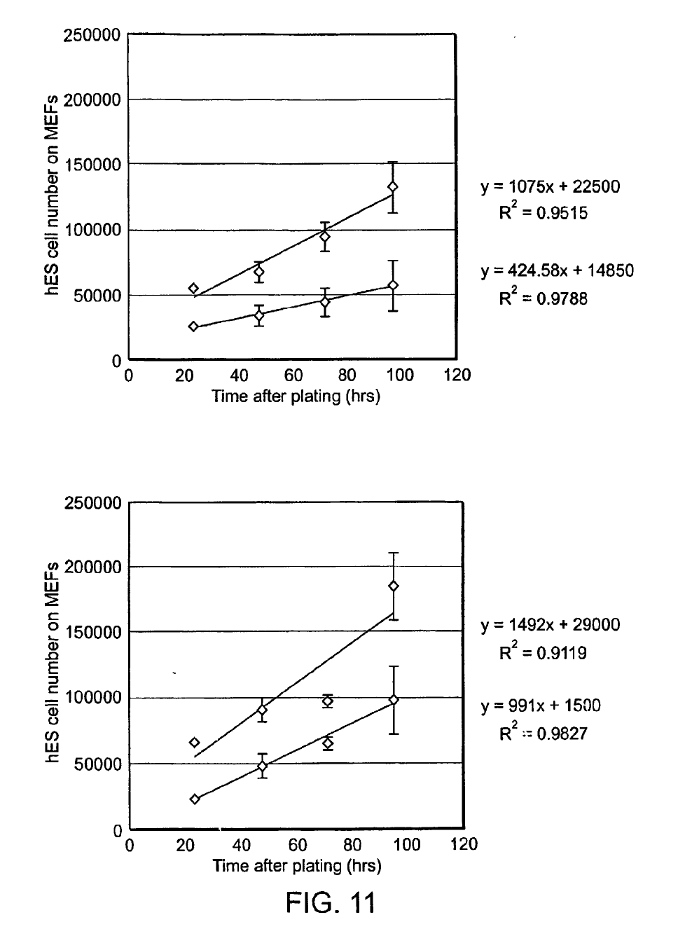

Using the hypothesis that factors required for hES cell survival would act through receptors present on the hES cell surface. Published hES cell microarray and SAGE data sets were searched for receptor tyrosine kinases expressed by hES cells that might act as receptors for anti-apoptotic factors⁹⁻¹¹. Notably, the information in these data sets suggests that hES cells might express TRKB and TRKC, the receptors for the nerve growth factor-related family of neurotrophins^{12 13}. As demonstrated in FIG. 1, TRKB and TRKC are expressed in hES by RT-PCR analysis, immunocytochemistry and western blotting (FIG. 1). TRKB and TRKC transcripts were present in both H1 and H9 hES cells, whereas transcripts for TRKA and the neurotrophin receptor, p75^{NGFR} were either absent or present at low levels (FIG. 1a and Supplementary FIG. 1). Immunostaining of H1 and H9 hES cells with antibodies to TRKA, TRKB and TRKC and p75^{NGFR} demonstrated the presence of TRKB and TRKC receptors on the cell surface of hES cells (FIG. 1b and data not shown). TRKA and p75^{NGFR} receptor proteins were either absent or present at low levels (FIG. 1b). Antibody specificity was confirmed by immunostaining cells that lack NT receptors (MEFs and HeLa cells) and cells that express NT receptors (primary neurons and PC12 cells) and also by immunostaining hES cells in the presence of a TRKB blocking peptide (FIG. 9). Western blotting of hES cell lysates was also carried out with antibodies to the TRKs as well as to p75^{NGFR}. In both H1 and H9 hES cells, TRKB and TRKC proteins were present, whereas TRKA and p75^{NGFR} were absent or present in much lower amounts (FIG. 9c).

### Example 2

### Effect of Neurotrophins on Clonal Survival of hES Cells

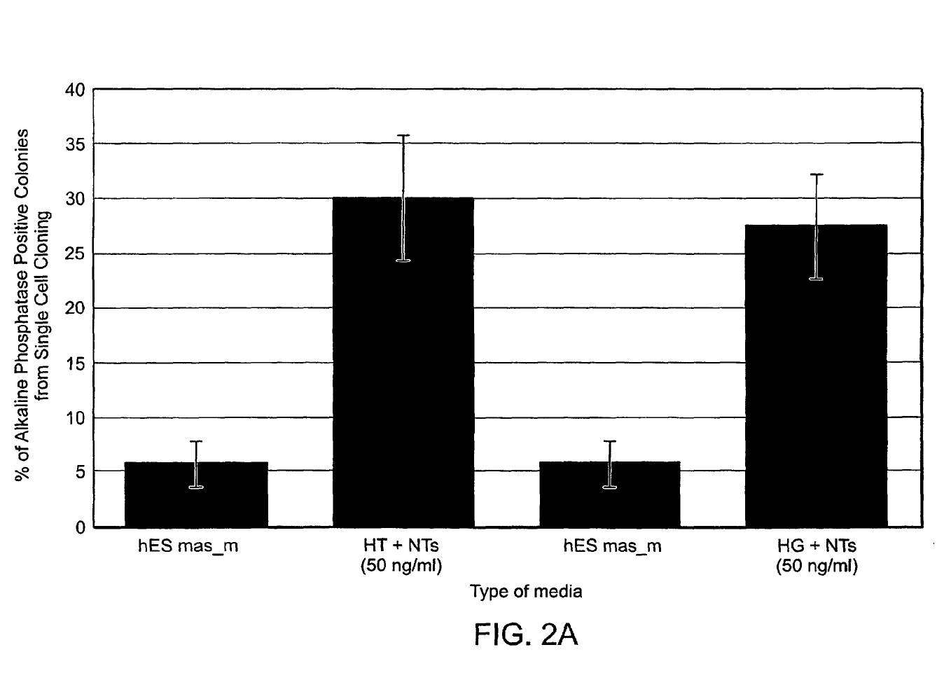

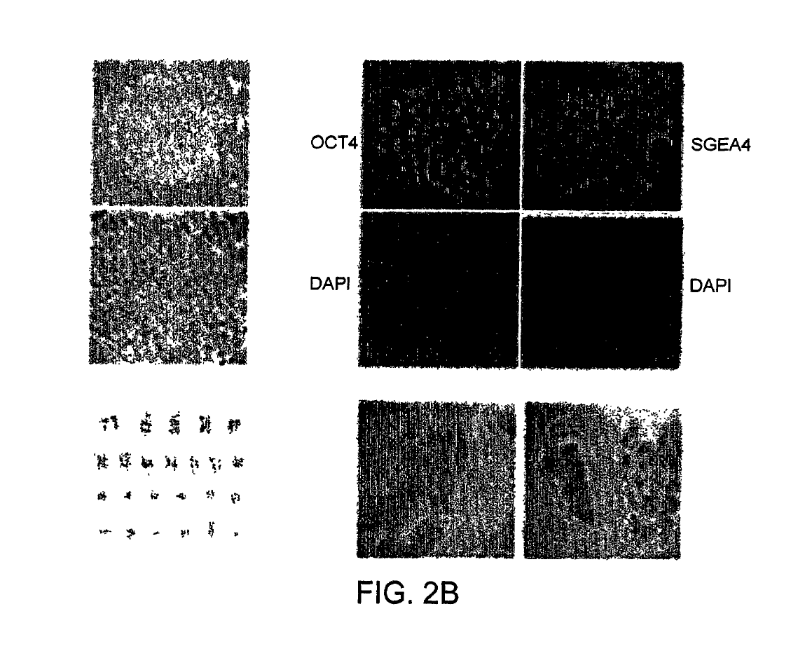

Because hES cells express both TRKB and TRKC at high levels, ligands for these receptors, BDNF, NT-3 and NT-4, were tested to determine their affect on clonal survival of hES cells. H1 and H9 hES cells were trypsinized and single cells were individually plated into wells of a 96-well plate containing hES medium with or without added neurotrophins (NTs: 50 ng/ml each BDNF, NT-3 and NT-4) and mitomycin-treated mouse embryo fibroblasts (MEFs) or Matrigel™. After 4 to 5 days, hES colonies were visualized by staining for alkaline phosphatase (AP), an activity characteristic of pluripotent stem cells. When grown in hES medium alone, about 6% of hES cells formed AP-positive colonies (FIG. 2a). In contrast, between 27 and 30% of hES cells grown in hES medium containing NTs formed AP-positive colonies (FIG. 2a). Thus, clonal survival of hES cells is significantly increased in the presence of neurotrophins. To test whether the effect of NTs on short-term hES cell clonal survival is reflected in the ability to derive clonal lines, we passaged clones derived in the presence or absence of NTs. Clones were passaged twice and the numbers of surviving clones counted. In the absence of NTs, 0.4% of the initial clones survived (Table 1), a number similar to that previously described for clonal derivation⁵. In the presence of NTs, 14.6% of the clones survived (Table 1).

TABLE 1

| | initial colony # | # of colonies picked | # survived P1 | # survived P2 | # of clones | cloning efficiency |
|---|---|---|---|---|---|---|
| # of hES colonies | 6 out of 96 | 6 | 0 | 0 | 0 | 2/480 = 0.4% |
| in hES media only | 7 out of 96 | 7 | 1 | 1 | 1 | |
| | 18 out of 288 | 18 | 1 | 1 | 1 | |
| # of hES colonies | 24 out of 96 | 12 | 5 | 5 | 5 × 24/12 = 10 | 42/288 = 14.6% |
| in hES + NTs | 25 out of 96 | 7 | 6 | 6 | 6 × 25/7 = 21 | |
| | 29 out of 96 | 18 | 7 | 7 | 7 × 29/18 = 11 | |

Table 1. Effect of neurotrophins on hES cell cloning efficiency. hES cells were plated as described in FIG. 2. After 4-5 days, clones (comprised of approximately 7-11 cells) were picked by manually scraping colonies out of each well in the 96 well plate. All colonies that were seen in regular hES cell medium were scraped (denoted passage 1 (P1)). Only a portion of the colonies grown in NTs were scraped because the colonies were so numerous. After another five days, the clones that survived were then passaged again by manual scraping (P2) and allowed to grow for a further 5 days which represents approximately 9 population doublings.

Therefore, addition of NTs results in a 36-fold increase in clonal survival. To determine whether hES cells maintained in NTs retain the characteristics of pluripotent hES cells, the expression of markers characteristic of pluripotent hES cells was tested and the differentiation potential of hES cells maintained in NTs. Four clonally-derived H1 hES cell lines were established and grown in the presence of NTs for 15 to 20 passages. Morphology characteristic of pluripotent hES cells was retained as well as expression of markers characteristic of pluripotent hES cells including OCT4, SSEA-3, SSEA-4, Tra-1-60, Tra-1-81 and AP (FIG. 2b and data not shown). In addition, the cells retained a normal, diploid karyotype. A total of 106 metaphase karyograms were examined for the four clonally-derived cell lines and all were normal (FIG. 2b). To demonstrate that hES cells cultured in NTs retain full developmental potency, their ability to make embryoid bodies in culture and to form teratomas was tested when injected into histocompatible mice. hES cells cultured in NTs formed well differentiated, cystic embryoid bodies identical to those obtained from hES cells cultured in standard conditions and formed teratomas containing differentiated cells derived from all three primary germ layers (data not shown and FIG. 2b). Because hES cells appear to differentiate easily into cells of the neuroectodermal lineage^{7,14}, hES cells cultured in NTs were also tested for expression of neuronal markers. hES cells grown with NTs do not express detectable amounts of the neuronal markers GFAP, MAP2, PAX2 or PAX6, indicating that they did not acquire a neuronal phenotype (Supplementary FIG. 2).

### Example 3

### Production of Neurotrophins by Mouse Embryonic Fibroblasts

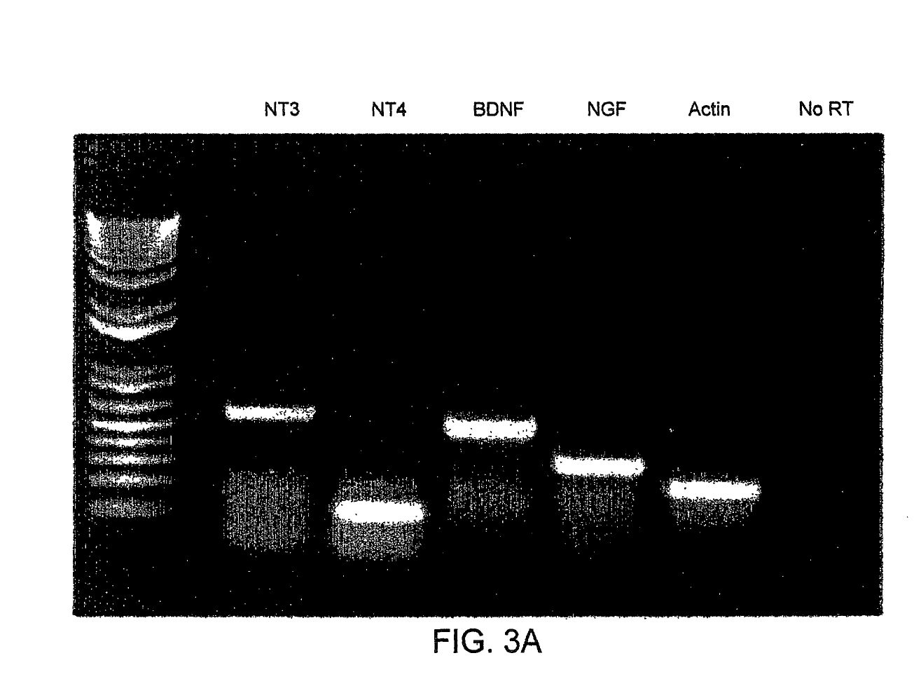

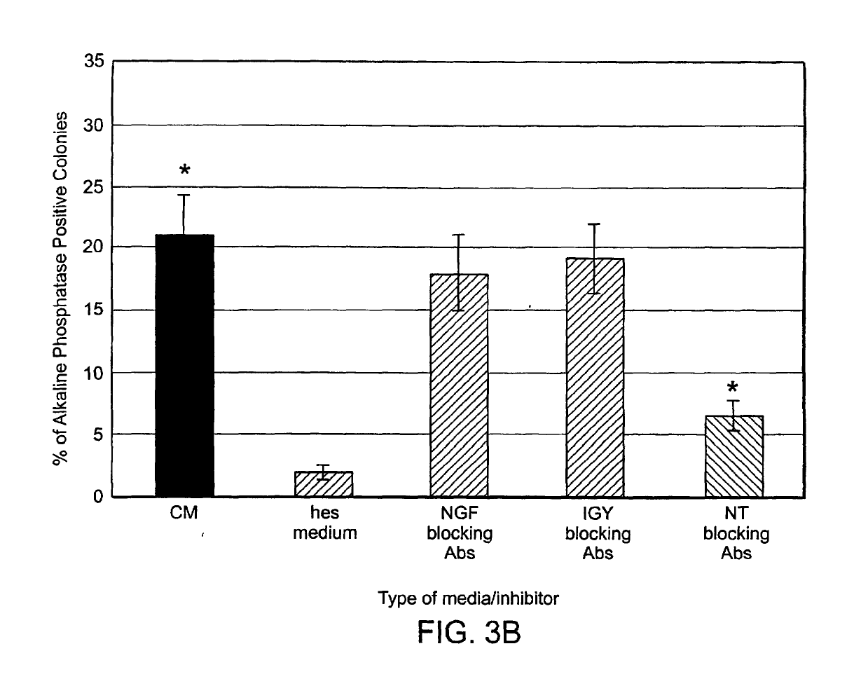

hES cells are typically grown on a feeder layer of mitotically inactivated MEFs or on Matrigel™ in the presence of medium conditioned by MBFs (MEF-CM)^{15 16}. The beneficial effects of MEF-CM might be due, at least in part, to the presence of neurotrophins. To test whether MEFs express neurotrophins, RT-PCR analysis was performed and demonstrated that MEFs express mRNA for NGF, BDNF, NT3 and NT4 (FIG. 3a). To test whether the beneficial effects of MEF-CM on hES growth is mediated by neurotrophins, the action of NTs was blocked with anti-neurotrophin antibodies in a low-density survival assay. In this assay, hES cells were dispersed to a single cell suspension by trypsin treatment and plated at low density (500 cells/well in a 96-well plate). They were cultured for 4 to 5 days in the presence of neurotrophin-neutralizing or control antibodies, then hES colonies visualized by AP staining (FIG. 3b). A combination of BDNF, NT3 and NT4 antibodies reduced cell survival from 21% to 6%, whereas an isotype-matched control antibody or an antibody to NGF had no significant effects (FIG. 3b). These data indicate that a portion of the survival activity of MEF-CM is indeed due to the action of neurotrophins.

### Example 4

### Neurotrophins Activate Tyrosine Kinase Expressed by hES Cells

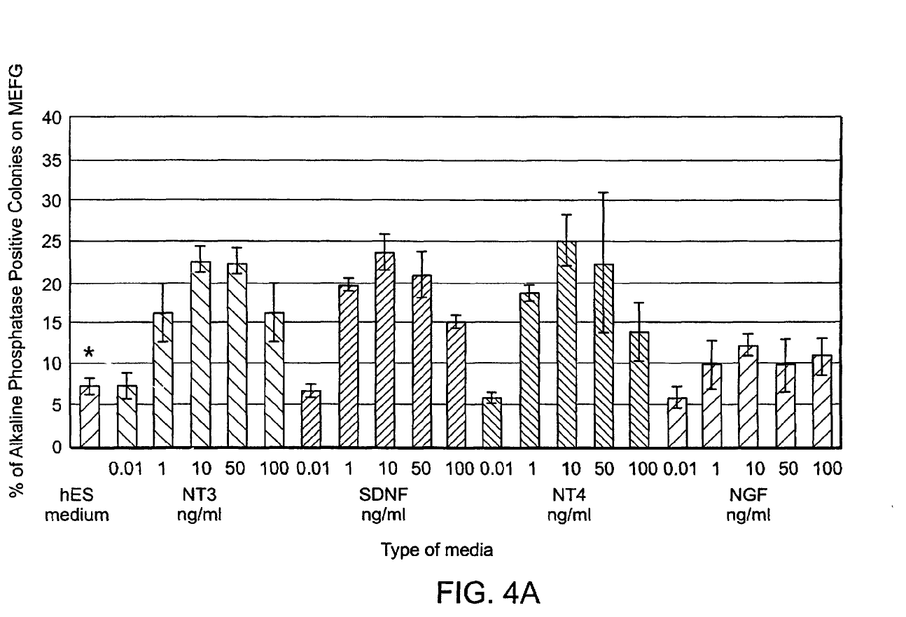

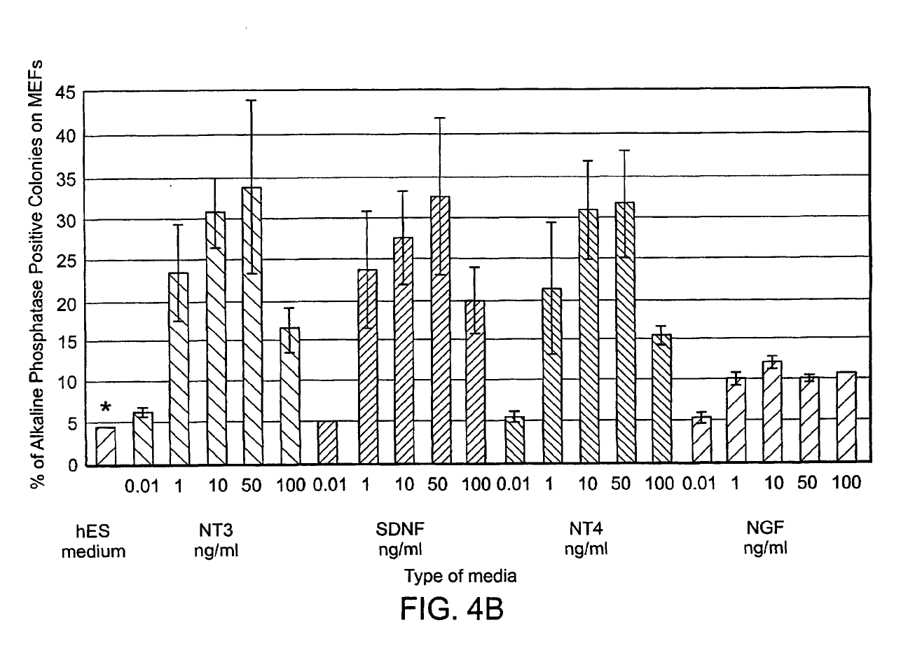

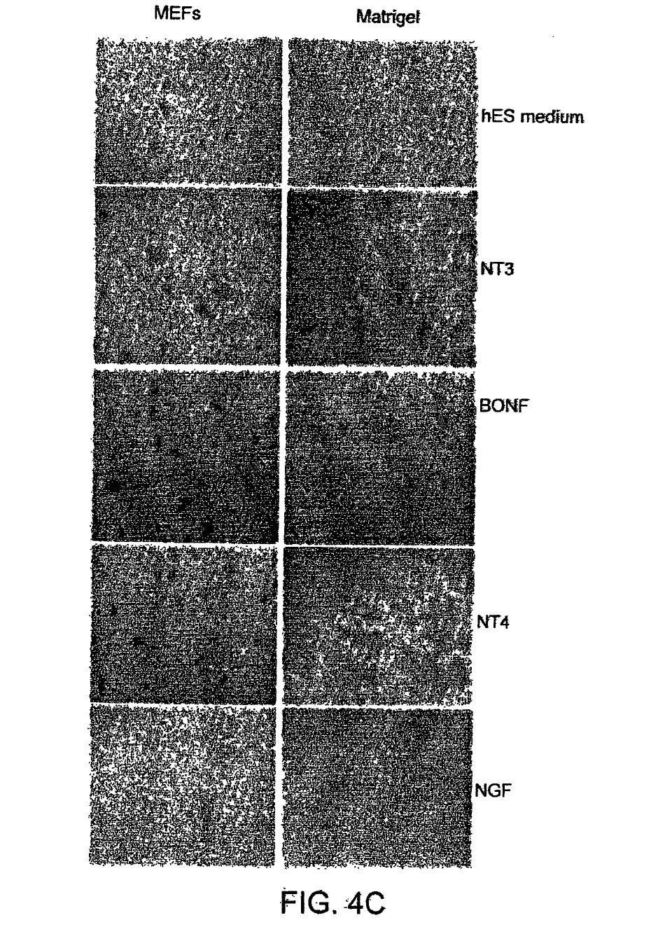

BDNF, NT3 and NT4 together act as survival factors for hES cells. To determine whether one or more of the neurotrophins alone can mediate survival of hES cells, NT-3, BDNF, NT-4 and NGF were tested individually in the low-density survival assay (FIG. 4). NT-3, BDNF and NT-4 had potent, dose dependent effects on hES cell survival when plated on either MEFs or Matrigel™ (FIG. 4). Furthermore, enhanced survival is observed in cells plated on Matrigel in the absence of MEFs suggesting that NTs act directly through TRK receptors on the hES rather than indirectly through MEFs. Addition of NGF resulted in a slight increase in hES cell survival (FIG. 4), possibly mediated by the low levels of TRKA and p75^{NGFR} present in hES cells. These data demonstrate that BDNF, NT-3 and NT4 are potent survival factors for hES cells acting presumably through TRKB and TRKC expressed by hES cells.

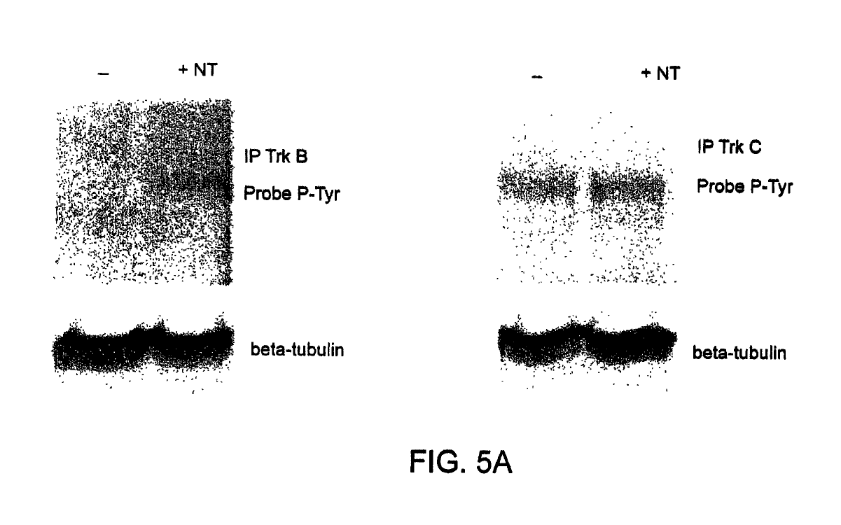

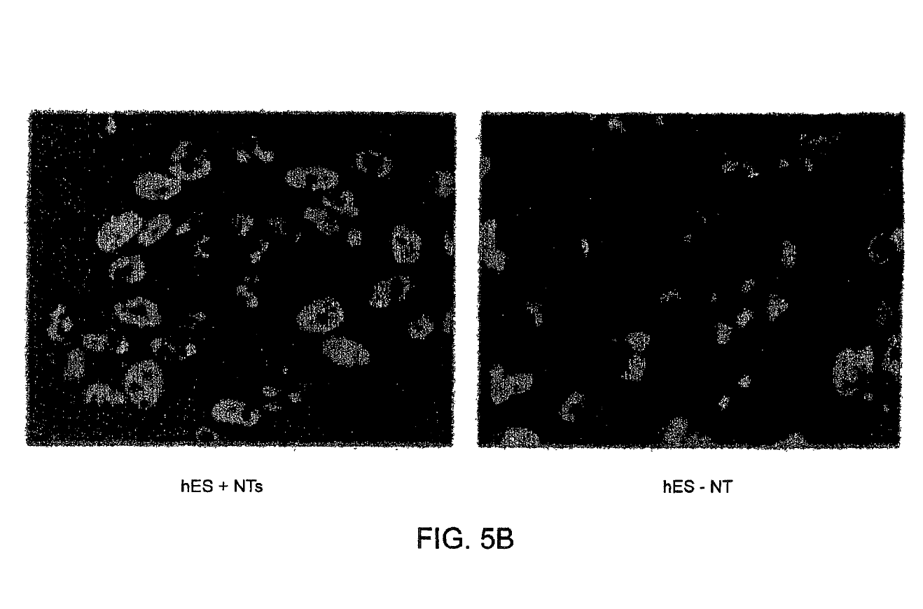

In neuronal cells, activation of the TRK receptors by neurotrophins results in their phosphorylation. To determine whether TRK receptors on hES cells are similarly activated by NTs, we analyzed the phosphorylation of TRKs in hES cells. TRK proteins were immunoprecipitated from hES cells with TRKB or TRKC antibodies and then blotted with an anti-phosphotyrosine (P-Tyr) antibody. In the absence of NTs, phosphorylated TRK proteins of approximately 145 Kda were present at low levels (FIG. 5a). When hES cells were exposed to NTs for 5 minutes, phosphorylation of TRKB and TRKC increased approximately 30 and 1.7 fold respectively (FIG. 5a), suggesting that TRKB and TRKC receptors on hES cells are activated in the presence of the NT3, BDNF and NT4. To test whether removal of NTs affects phosphorylation of TRK proteins in hES cells grown continuously in NTs. For this study, we visualized TRK phosphorylation immunocytochemically using an antibody that binds to TRK proteins phosphorylated at tyrosine 490 (P-TRK(490)). In the presence of NTs, P-TRK(490) stained hES cells throughout the colonies (FIG. 5b). When NTs were removed from the medium for 20 minutes, P-TRK(490) staining disappeared from many of the cells (FIG. 5b and Supplementary FIG. 2) confirming that NT3, BDNF and NT4 affect TRK phosphorylation in hES cells. Notably, P-TRK(490) staining is often lost from the center of colonies, the area of hES cell colonies that is often seen to undergo apoptosis or differentiation (FIG. 5b and Supplementary FIG. 2)⁴. Anti-P-TRK antibodies also stained hES cells growing on MEFs, supporting the idea that part of the effect of MEF-CM is mediated through activation of the Trk receptors by NTs produced by MEFs.

### Example 5

### Inhibition of Tyrosine Kinase Signaling Reduces hES Cell Survival

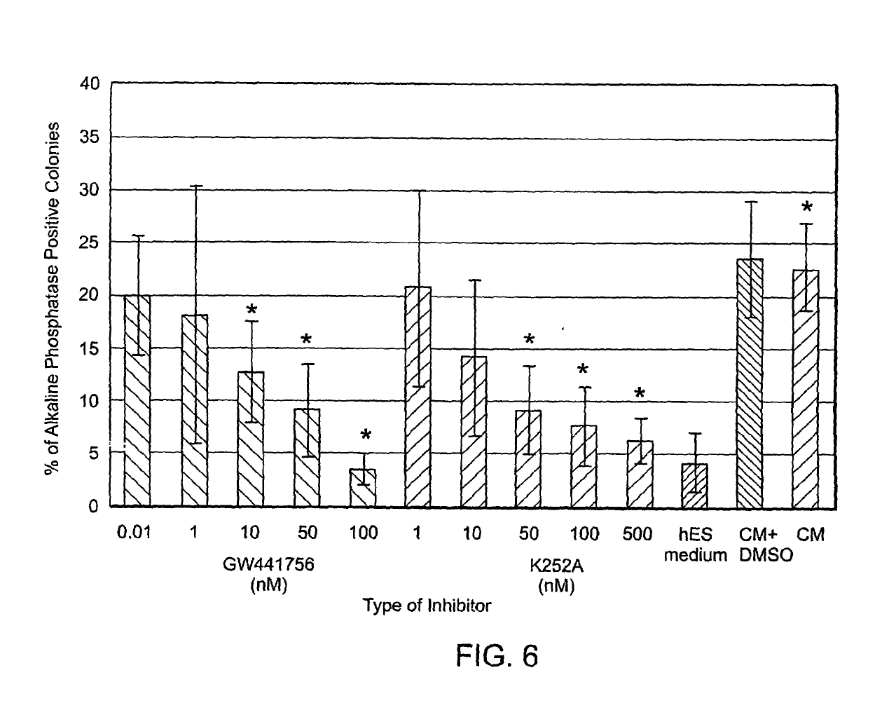

If neurotrophins are mediating hES cell survival through activation of TRK signaling pathways, then pharmacological inhibition of TRK signaling pathway should reduce hES cell survival. To test this prediction, hES cell survival was measured using the low density survival assay in the presence or absence of two different TRK inhibitors, GW441756 and K252a¹⁷⁻¹⁹. K252a is a staurosporine analog that has a broad kinase inhibition profile, including potent inhibition of TRKs. K252a has been widely used as a TRK inhibitor with specificity for TRKs over FGF, EGF and other signaling pathways previously demonstrated¹³. GW441756 is a 3-anilinomethylene-oxindole analog with a more specific kinase inhibition profile (see compound #3 in¹⁹). In a kinase inhibition assay, this compound has 10-fold, or in some cases 100-fold, selectivity for TRKs over many kinases including c-Src, VEGFR2, Raf and CDK1¹⁹. Both GW441756, which binds to the ATP binding site of the TRK receptors, and K252a, which inhibits tyrosine phosphorylation and kinase activity of TRKS, showed statistically-significant and dose-dependent effects on hES cell survival (FIG. 6). Although pharmacological inhibitors can often affect multiple signaling pathways, the demonstration that two TRK inhibitors, working through different mechanisms, have the same effect on hES cell survival suggests the effect observed is mediated through blocking the action of TRKS. These results demonstrate that the TRK receptors are not only present and activated in response to neurotrophins but blocking their action reduces hES cell survival. Taken together with the ability of NT-neutralizing antibodies to block hES cell survival (FIG. 3b), these data suggest an important role for NT-mediated activation of TRK signaling in hES cell survival.

### Example 6

### Neurotrophins Mediate hES Survival by Decreasing Apoptosis

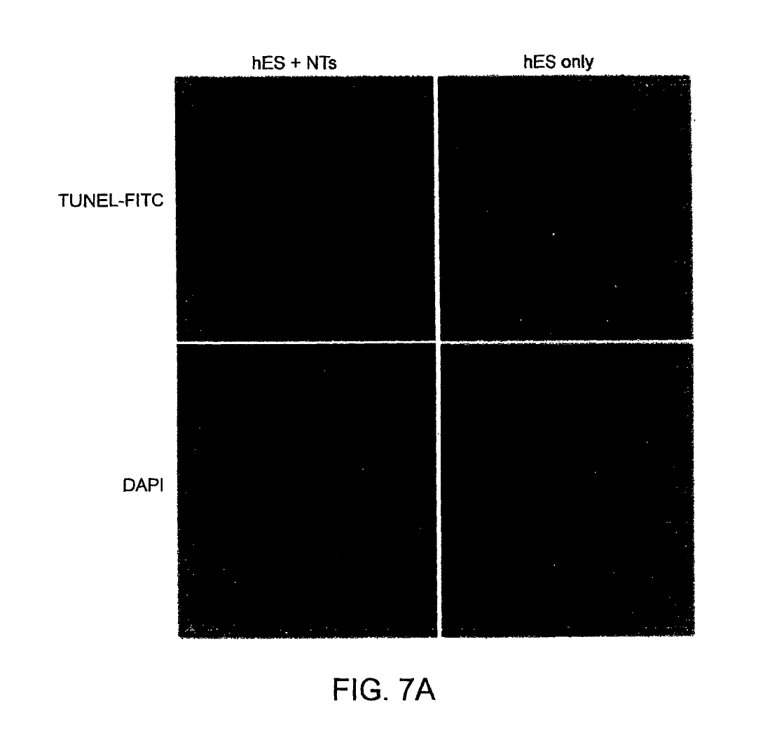

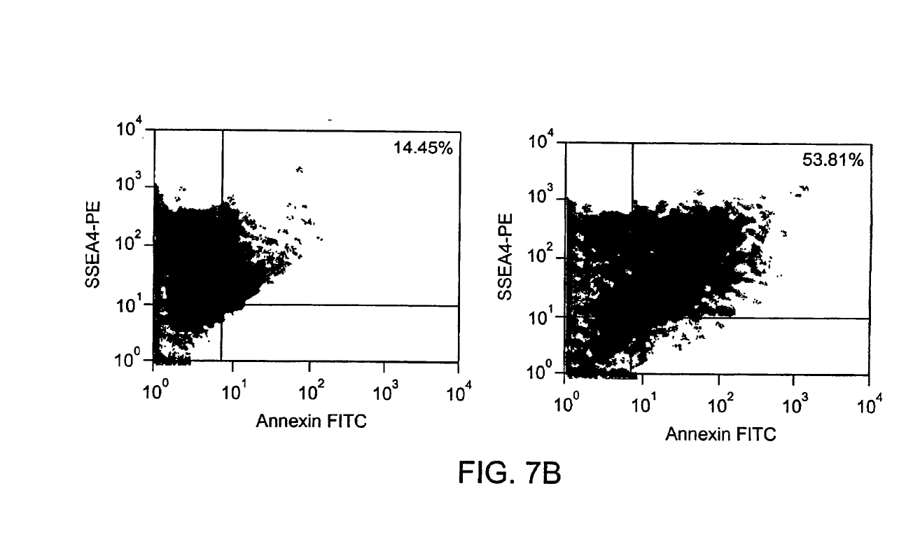

Whether the increase in clonal hES survival mediated by TRKs is associated with a decrease in apoptosis was also examined. Clumps of hES cells were plated on Matrigel in the presence or absence of NTs. The surviving colonies were analyzed by TUNEL staining. In the absence of NTs, a large number of TUNEL-positive apoptotic cells were observed in colonies of hES cells (FIG. 7a). In contrast, few TUNEL-positive cells were observed in hES cells plated in the presence of neurotrophins (FIG. 7a). Flow cytometry was used to quantify apoptosis in hES cells. Clumps of hES cells grown on Matrigel™ in the presence or absence of neurotrophins for 24 hours were stained with the early apoptotic marker Annexin V-FITC and the hES cell marker SSEA-4. A significantly greater proportion of hES cells grown without NTs labeled with Annexin V-FITC (53.81%) than hES cells grown with NTs (14.45%; FIG. 7b). Similar results from both TUNEL staining and flow cytometry were observed when trypsin-dispersed hES cells were plated as single cells at low-density in the presence or absence of NTs (data not shown). These data suggest that hES cells grown without NTs undergo increased apoptosis. In contrast, growth in neurotrophins significantly decreases both TUNEL-positive and Annexin V-FITC-positive populations of hES cells indicating that neurotrophins act in an anti-apoptotic fashion to promote hES cell survival. Because NTs can act to stimulate DNA synthesis in some cells 12, we also examined the mitotic index in cells grown with or without NTs. No differences in mitotic index were observed (data not shown). In contrast, we did observe a decrease in the population doubling time of hES cell cultured in the presence of NTs (Supplementary. FIG. 3) most likely due to the suppression of hES cell apoptosis by NTs. The decrease in population doubling time was more pronounced in hES cells grown on Matrigel than on MEFs, presumably because MEFs produce some NTs (see FIG. 3) and the addition of NTs, therefore, has less effect (Supplementary. FIG. 3). In both conditions (Matrigel and MEFs) NTs improve the initial bulk survival of trypsinized hES cells in agreement with the effect seen on clonal cell survival. The combined action of NTs on initial survival and on population doubling time of hES cells together results in a significant improvement in their expansion in culture (Supplementary. FIG. 3).

### Example 7

### Survival Effect of Neurotrophins is Mediated through TRK Activation of the PI-3K Pathwav

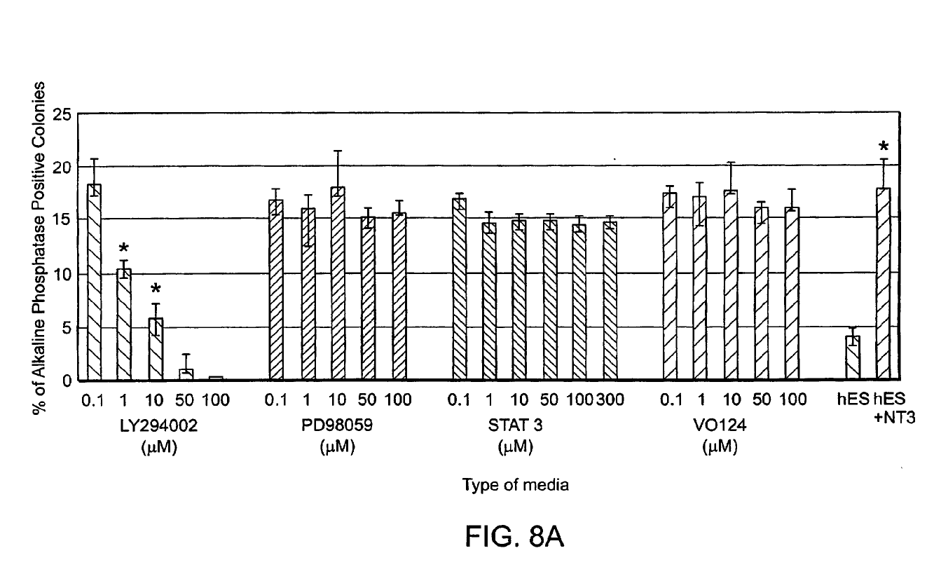

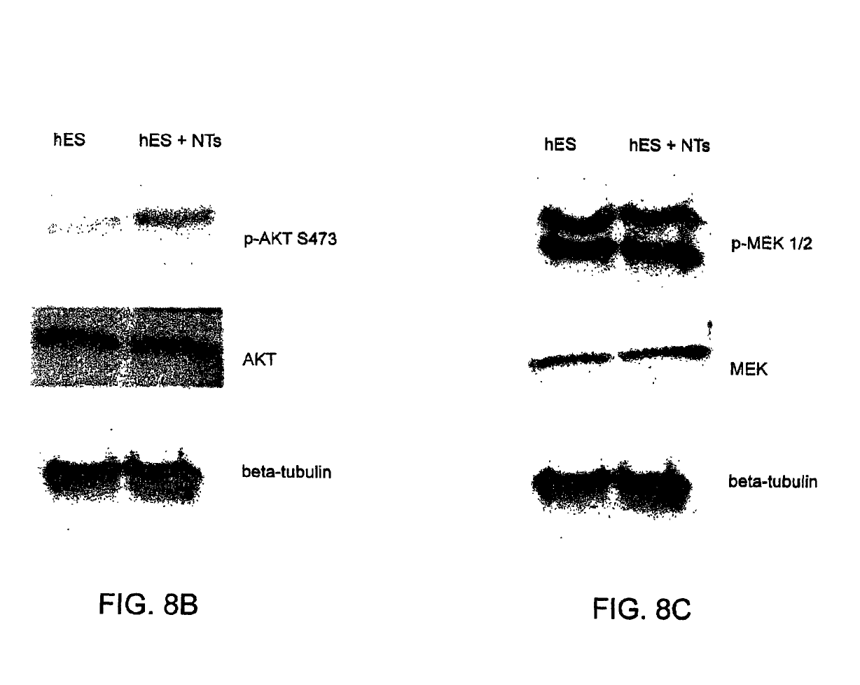

Activation and phosphorylation of TRK receptors leads to activation of a number of downstream effectors including phosphatidylinositol-3-kinase (PI-3K) and mitogen-activated protein kinase (MAPK). To test the role of these molecules in neurotrophin-mediated survival of hES cells we perturbed their action with pharmacological inhibitors. As a control we used inhibitors to the JAK/STAT signaling pathway, a pathway known to have little, if any, role in hES cell growth^{20 21}. bFGF was omitted in these cultures in order to eliminate any effects resulting from inhibition of bFGF signaling. Inhibition of the PI-3K pathway had a dramatic effect on hES cell survival in neurotrophins, whereas inhibition of the MAPK and JAK/STAT pathways had little or no effect on hES cell survival (FIG. 8a). Consistent with the observed effects of PI-3K and MAPK inhibitors of hES cell survival, we observed that phosphorylation of AKT, a downstream effector of PI-3K, increases upon addition of NTs to hES cells (FIG. 8a, b). In contrast, phosphorylation of MEK1/2, a downstream effector of MAPK, was unaffected by the addition of NTs. These data suggest that activation of PI-3K, presumably acting through AKT, is a critical event in neurotrophin-mediated hES cell survival.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

1. An aqueous composition for culturing human embryonic stem (hES) cells in vitro comprising a culture medium supplemented with added exogenous neurotrophins in an effective concentration to increase the survival of the hES cells cultured in the neurotrophin supplemented culture medium, relative to the survival of hES cells cultured in an unsupplemented culture medium.

2. A culture media of claim 1 wherein the neurotrophins are selected from the group consisting of brain-derived neurotophic factor (BDNF), neurotrophin-3 (NT-3) and neurotrophin-4 (NT-4).

3. A method of culturing human hES cells comprising culturing the stem cells in the neurotrophin supplemented aqueous composition of claim 1.

4. A method of claim 3 wherein the neurotrophins are selected from the group consisting of brain-derived neurotophic factor (BDNF), neurotrophin-3 (NT-3) and neurotrophin-4 (NT-4).

5. A method of increasing the survival of hES cells comprising culturing the stem cells in the neurotrophin supplemented aqueous composition of claim 1.

6. A method of claim 5 wherein the neurotrophins are selected from the group consisting of brain-derived neurotophic factor (BDNF), neurotrophin-3 (NT-3) and neurotrophin-4 (NT-4).

7. A method of increasing the proliferation of human embryonic stem cells comprising culturing the stem cells in the neurotrophin supplemented aqueous composition of claim 1.

8. A method of claim 7 wherein the neurotrophins are selected from the group consisting of brain-derived neurotophic factor (BDNF), neurotrophin-3 (NT-3) and neurotrophin-4 (NT-4).

9. An aqueous composition for culturing primate embryos in vitro comprising a culture medium capable of supporting embryo development supplemented with exogenous neurotrophins in an effective concentration to increase the survival of viable primate embryos cultured in the neurotrophin supplemented culture medium, relative to the survival of embyros cultured in an unsupplemented culture medium.

10. An aqueous composition of claim 9 wherein the neurotrophins are selected from the group consisting of brain-derived neurotophic factor (BDNF), neurotrophin-3 (NT-3) and neurotrophin-4 (NT-4).

11. A method of increasing the survival of a primate embryo following in vitro fertilization comprising incubating the embryo in a culture media containing neurotrophins.

12. A method of carrying out in vitro fertilization of a primate oocyte and achievement of pregnancy in a receptive female according to the following steps: inducing hyperovulation by hormonal therapy, retrieving and incubating the oocytes in vitro in a culture media, fertilizing the oocytes with freshly obtained, capacitated sperm to obtain primate embryos, incubating the primate embryos in vitro in a culture media supplemented with neurotrophins in an effective concentration to increase survival from the time of double pronuclei visualization to the implantation stage of the mature blastocyst, harvesting the blastocyst from the culture media and releasing it into a physiologically receptive uterus.

13. A method of claim 11 wherein the neurotrophins are selected from the group consisting of brain-derived neurotophic factor (BDNF), neurotrophin-3 (NT-3) and neurotrophin-4 (NT-4).

14. A method of claim 11 wherein the primate is human.

15. A method for arresting embryo development in primates in which a neurotrophin antagonist is administered intravenously, intramuscularly, or transdermally in a dose sufficient to decrease embryo survival.

16. A method of contraception in a female primate comprising intravenous, intramuscular, or transdermal administration to the primate a neurotrophin antagonist.

17. A method of claim 15 wherein the primate is human.

18. A method of claim 15 wherein the neurotrophin antagonist is an antibody directed to a neurotrophin are selected from the group consisting of brain-derived neurotophic factor (BDNF), neurotrophin-3 (NT-3) and neurotrophin-4 (NT-4).

19. A method of arresting hES cell survival in vitro comprising culturing the hES cells in media containing a neurotrophin antagonist.

20. A method of arresting embryo survival in vitro comprising culturing the embryo in media containing a neurotrophin antagonist.
